SEQUENCE LISTING (21 sequences)

SEQ ID NO: 1 - DNA, 53 bp, unidentified
  source 1..53: mol_type = other DNA
  modified_base >53: mod_base = OTHER; note = 3'-end modification: two inverted thymidines
tcttgcgtatcttctccacctcagcgcaagatcaacatcagtctgataagcta

SEQ ID NO: 2 - DNA, 53 bp, synthetic construct
  source 1..53: mol_type = other DNA
  modified_base 38..39: mod_base = OTHER; note = Abasic site
  modified_base >53: mod_base = OTHER; note = 3'-end modification: two inverted thymidines
tcttgcgtatcttctccacctcagcgcaagatcaacannagtctgataagcta

SEQ ID NO: 3 - DNA, 53 bp, synthetic construct; identical to SEQ ID NO: 2
  source 1..53: mol_type = other DNA
  modified_base 38..39: mod_base = OTHER; note = Ethynyl abasic site
  modified_base >53: mod_base = OTHER; note = 3'-end modification: two inverted thymidines

SEQ ID NO: 4 - DNA, 53 bp, synthetic construct
  source 1..53: mol_type = other DNA
  modified_base 38: mod_base = OTHER; note = Abasic site
  modified_base >53: mod_base = OTHER; note = 3'-end modification: two inverted thymidines
tcttgcgtatcttctccacctcagcgcaagatcaacancagtctgataagcta

SEQ ID NO: 5 - DNA, 53 bp, synthetic construct
  source 1..53: mol_type = other DNA
  modified_base 38..40: mod_base = OTHER; note = Abasic site
  modified_base >53: mod_base = OTHER; note = 3'-end modification: two inverted thymidines
tcttgcgtatcttctccacctcagcgcaagatcaacannngtctgataagcta

SEQ ID NO: 6 - DNA, 53 bp, synthetic construct
  source 1..53: mol_type = other DNA
  modified_base 38..41: mod_base = OTHER; note = Abasic site
  modified_base >53: mod_base = OTHER; note = 3'-end modification: two inverted thymidines
tcttgcgtatcttctccacctcagcgcaagatcaacannnntctgataagcta

SEQ ID NO: 7 - DNA, 53 bp, synthetic construct
  source 1..53: mol_type = other DNA
  modified_base 40..41: mod_base = OTHER; note = Abasic site
  modified_base >53: mod_base = OTHER; note = 3'-end modification: two inverted thymidines
tcttgcgtatcttctccacctcagcgcaagatcaacatcnntctgataagcta

SEQ ID NO: 8 - DNA, 53 bp, synthetic construct
  source 1..53: mol_type = other DNA
  modified_base 39..40: mod_base = OTHER; note = Abasic site
  modified_base >53: mod_base = OTHER; note = 3'-end modification: two inverted thymidines
tcttgcgtatcttctccacctcagcgcaagatcaacatnngtctgataagcta

SEQ ID NO: 9 - DNA, 53 bp, synthetic construct
  source 1..53: mol_type = other DNA
  modified_base 37..38: mod_base = OTHER; note = Abasic site
  modified_base >53: mod_base = OTHER; note = 3'-end modification: two inverted thymidines
tcttgcgtatcttctccacctcagcgcaagatcaacnncagtctgataagcta

SEQ ID NO: 10 - DNA, 53 bp, synthetic construct
  source 1..53: mol_type = other DNA
  modified_base 36..37: mod_base = OTHER; note = Abasic site
  modified_base >53: mod_base = OTHER; note = 3'-end modification: two inverted thymidines
tcttgcgtatcttctccacctcagcgcaagatcaanntcagtctgataagcta

SEQ ID NO: 11 - DNA, 53 bp, synthetic construct
  source 1..53: mol_type = other DNA
  modified_base 35..36: mod_base = OTHER; note = Abasic site
  modified_base >53: mod_base = OTHER; note = 3'-end modification: two inverted thymidines
tcttgcgtatcttctccacctcagcgcaagatcannatcagtctgataagcta

SEQ ID NO: 12 - DNA, 49 bp, synthetic construct
  source 1..49: mol_type = other DNA
  modified_base 38..39: mod_base = OTHER; note = Abasic note
  modified_base >49: mod_base = OTHER; note = 3'-end modification: two inverted thymidines
agcagcaacatagcagaacctcagcgctgcttcttgcnnatcttctcca

SEQ ID NO: 13 - DNA, 65 bp, synthetic construct
  source 1..65: mol_type = other DNA
  modified_base 50..51: mod_base = OTHER; note = Abasic note
  modified_base >65: mod_base = OTHER; note = 3'-end modification: two inverted thymidines
agcagcaacatagcagaacctcagcttctgctatgttgctgcttcaacannagtctgataagcta

SEQ ID NO: 14 - DNA, 53 bp, synthetic construct
  source 1..53: mol_type = other DNA
  modified_base 41..42: mod_base = OTHER; note = Abasic note
  modified_base >53: mod_base = OTHER; note = 3'-end modification: two inverted thymidines
tcttgcgtatcttctccacctcagcatacgcaagaagcagnnacatagcagaa

SEQ ID NO: 15 - DNA, 65 bp, synthetic construct
  source 1..65: mol_type = other DNA
  modified_base >65: mod_base = OTHER; note = 3'-end modification: two inverted thymidines
agcagcaacatagcagaacctcagcttctgctatgttgctgcttcaacataagtctgataagcta

SEQ ID NO: 16 - DNA, 53 bp, synthetic construct
  source 1..53: mol_type = other DNA
  modified_base >53: mod_base = OTHER; note = 3'-end modification: two inverted thymidines
tcttgcgtatcttctccacctcagcatacgcaagaagcagcaacatagcagaa

SEQ ID NO: 17 - DNA, 57 bp, synthetic construct
  source 1..57: mol_type = other DNA
  modified_base 42..43: mod_base = OTHER; note = Abasic note
tcttgcgtatcttctccacctcagcatacgcaagatcaacannagtctgataagcta

SEQ ID NO: 18 - DNA, 61 bp, synthetic construct
  source 1..61: mol_type = other DNA
  modified_base 46..47: mod_base = OTHER; note = Abasic note
  modified_base >61: mod_base = OTHER; note = 3'-end modification: two inverted thymidines
tcttgcgtatcttctccacctcagcgaagatacgcaagatcaacannagtctgataagcta

SEQ ID NO: 19 - DNA, 65 bp, synthetic construct
  source 1..65: mol_type = other DNA
  modified_base 50..51: mod_base = OTHER; note = Abasic note
  modified_base >65: mod_base = OTHER; note = 3'-end modification: two inverted thymidines
tcttgcgtatcttctccacctcagctggagaagatacgcaagatcaacannagtctgataagcta

SEQ ID NO: 20 - DNA, 22 bp, synthetic construct
  source 1..22: mol_type = other DNA
tagcttatcagactgatgttga

SEQ ID NO: 21 - RNA, 22 nt, synthetic construct
  source 1..22: mol_type = other RNA
tagcttatcagactgatgttga

(19) United States
(12) Patent Application Publication
(10) Pub. No.: US 2024/0117414 A1
(43) Pub. Date: Apr. 11, 2024

(54) SEQUENCE CONVERSION AND SIGNAL AMPLIFIER DNA HAVING ABASIC NUCLEIC ACIDS, AND DETECTION METHODS USING SAME

(71) Applicant: ABBOTT LABORATORIES, Abbott Park, IL (US)

(72) Inventors: Takamitsu Morikawa, Matsudo-City (JP); Toru Yoshimura, Matsudo-City (JP)

(21) Appl. No.: 18/362,013

(22) Filed: Jul. 31, 2023

Related U.S. Application Data
(63) Continuation of application No. PCT/US2022/014590, filed on Jan. 31, 2022.
(60) Provisional application No. 63/144,146, filed on Feb. 1, 2021.

(51) Int. Cl. C12Q 1/6816

(52) U.S. Cl. CPC C12Q 1/6816; C12Q 2600/166

(57) ABSTRACT

Disclosed are methods for detecting a target nucleic acid in a sample. The methods include contacting the sample, in the presence of a polymerase and an endonuclease, with a first oligonucleotide that includes, in the 5′ to 3′ direction, a signal DNA generation sequence, an endonuclease recognition site, and a complementary sequence that has at least one abasic moiety and wherein the complementary sequence has a first complementary sequence that is complementary to at least a portion of the signal DNA generation sequence and a second complementary sequence that is complementary to the 3′ end of the target nucleic acid. Also disclosed are methods that include a second oligonucleotide including, in the 5′ to 3′ direction, a second signal DNA generation sequence, an endonuclease recognition site, and a sequence that is homologous to the first signal DNA generation sequence of the first oligonucleotide and that optionally has at least one abasic site. Also disclosed chemically modified oligonucleotides, as well as compositions and kits that include the chemically modified oligonucleotides for detecting a target nucleic acid.

### CROSS-REFERENCE TO RELATED APPLICATIONS

This application is a continuation application of International Patent Application serial number PCT/US2022/014590, filed Jan. 31, 2022, which is related to and claims the benefit of priority from U.S. Provisional patent application Ser. No. 63/144,146, filed Feb. 1, 2021, and which are hereby incorporated by reference in their entirety.

### SEQUENCE LISTING

This application contains a Sequence Listing which is incorporated by reference and is submitted with the filing of this application in computer readable format entitled, “12835US01_60982US02_SeqList.XML”. The Sequence Listing file was created on Nov. 20, 2023 and is 39,339 bytes in size. The XML file also serves as any paper copy of the Sequence Listing that may be required for purposes of sequence listing submissions.

### FUNDING

[Not Applicable]

### BACKGROUND

The detection of target nucleic acid in test samples is important in various fields, including medicine and biology. Many compositions, assay platforms, and procedures are available for the detection of specific nucleic acid molecules. In order for detection to be reproducible and accurate, these procedures require no or low levels of non-specific background amplification. However, amplification methods may give rise to false positive signals that affect the quality, accuracy, reproducibility, and overall reliability of the results. In some assays these “false” positive signals can be detected in samples, including control samples that contain non-template DNA (non-target DNA) or even samples that lack any DNA template.

One common method used for amplification of specific sequences from a population of mixed nucleic acid sequences is the polymerase chain reaction (PCR). Since a typical PCR is carried out at three different temperatures, the reaction can be associated with challenges such as difficulty in maintaining accurate temperatures and that the time loss increases in proportion to the number of amplification cycles. The denaturation of a double-stranded template DNA into single strands (while dependent to some extent on the particular sequence) often requires the use of high “melting” temperatures, which limits the class of DNA polymerases that can be used to those that are highly thermostable. Consequently, isothermal amplification platform technologies have been developed to detect nucleic acids under reaction conditions that are milder than those used in PCR. Nevertheless, these isothermal amplification technologies have not addressed the challenges that are presented by non-specific amplification events and high background signals that can interfere with target sequence detection.

The following disclosure provides alternative methods and compositions for detecting a nucleic acid sequence (such as DNA or RNA) under reaction conditions that are less rigorous than those used in PCR. The methods and compositions maintain sequence selectivity and sensitivity that allow for the detection of nucleic acid molecules that may be in a sample at low concentrations and/or nucleic acid molecules of a short length. The methods and compositions also reduce any background signal that may result from non-specific and/or target-independent amplification events. Among other aspects, the disclosure provides novel methods, reagents, and nucleic acid molecules that can improve the detection limit of target nucleic acids in a sample under low temperature, isothermal conditions, and can simplify or improve sample preparation and automated methods of detection.

### SUMMARY OF THE INVENTION

In one aspect, the disclosure relates to a method for detecting a target nucleic acid in a sample, said method comprising contacting said sample with: a covered (i.e., hairpin) oligonucleotide (sequence conversion DNA or SC DNA) comprising, in the 5′ to 3′ direction, a signal DNA generation sequence (A), an endonuclease recognition site (B), and a complementary sequence comprising a first complementary sequence (C) that is complementary to at least a portion of the signal DNA generation sequence, and a second complementary sequence (D) having at least one abasic site (or “abasic group”, “abasic moiety”, or “abasic residue”) incorporated therein and which is complementary to the 3′ end of a target nucleic acid; a polymerase; and an endonuclease for a nicking reaction. In embodiments of this aspect, the method also comprises determining the presence or absence of a signal DNA, wherein the presence of the signal DNA indicates the presence of the target nucleic acid in the sample.

In one aspect, the disclosure relates to methods for detecting a target nucleic acid in a sample, wherein the interaction of a target nucleic acid with a first oligonucleotide (sequence conversion DNA or SC DNA) that has one or more abasic sites primes replication by a polymerase to produce a first signal DNA (S1) that in turn interacts with a second oligonucleotide (signal amplifier DNA or cascade signal amplifier DNA 1 or cSA DNA 1), that may have one or more abasic moiety (or site), and primes replication by a polymerase to produce a second signal DNA (S2) different from the first signal DNA, S1, which in turn can interact with a third oligonucleotide (cascade signal amplifier DNA 2 or cSA DNA 2), that may have one or more abasic sites, and primes replication by a polymerase to produce a third unique signal DNA (S3). The third unique signal DNA S3 can interact with a fourth oligonucleotide (cascade signal amplifier DNA 3 or cSA DNA 3), that may have one or more abasic sites, and primes replication by a polymerase to produce a fourth unique signal DNA (S4), which in turn can interact with a fifth oligonucleotide (cascade signal amplifier DNA 4 or cSA DNA 4), that may have one or more abasic sites, and primes replication by a polymerase to produce a fifth unique signal DNA S5, which in turn can interact with a sixth oligonucleotide (cascade signal amplifier DNA 5 or cSA DNA 5), that may have one or more abasic sites, and primes replication by a polymerase to produce a sixth unique signal DNA S5, and so on until the desired amplification is reached. In various embodiments of these aspects, the abasic site can be positioned in the nucleotide sequence toward the 3′ half of the sequence (e.g., within a portion of the oligonucleotide sequence that is closer to, or within, the 3′ end of the SC or cSA DNAs that hybridize with a target or a signal DNA). In further embodiments, the abasic site(s) can be located at position 3, 4, 5, 6, 7, 8, 9, 10, 11, 12, 13, 14, 15 or a combination thereof from the first nucleotide at the 5′ end of the sequence that is complementary to the 3′ end of the target nucleic acid of said first oligonucleotide (e.g., sequence (D) referred to above). Some embodiments may describe the abasic site(s) as located at a position that is 3, 4, 5, 6, 7, 8, 9, 10, 11, 12, 13, 14, or 15 nucleotides from the 3′ end of the endonuclease recognition site, or located at a combination of those positions.

For example, in some embodiments of the various aspects described herein, the disclosure provides a method (as well as compositions, kits, and chemically modified oligonucleotides) for detecting a target nucleic acid in a sample, said method comprising contacting said sample with: a first oligonucleotide (which may be identified herein as a sequence conversion DNA or SC DNA) comprising, in the 5′ to 3′ direction, a first signal DNA generation sequence (A), an endonuclease recognition site (B), and a complementary sequence that comprises a first complementary sequence (C) that is complementary to at least a portion of the signal DNA generation sequence (A) and a second complementary sequence (D) that is complementary to the 3′ end of the target nucleic acid, with the sequence (D) having at least one abasic site (or “abasic group”, “abasic moiety”, or “abasic residue”) incorporated therein, wherein at least a portion of the first signal DNA generation sequence (A) and the first complementary sequence (C) that is complementary to the signal DNA generation sequence (A) hybridize to form a hairpin structure. In some further embodiments, the methods, compositions, and kits comprise a second oligonucleotide (which may be identified herein as a signal amplifier DNA or a cascade signal amplifier DNA 1 (SA DNA or cSA DNA 1) comprising, in the 5′ to 3′ direction, a second unique signal DNA generation sequence (E), an endonuclease recognition site (F) (which may be the same or different from the endonuclease recognition site (B) in the SC DNA), a sequence (G) that is complementary to at least a portion of the signal DNA generation sequence (E), and a sequence (H) that is homologous to the first signal DNA generation sequence (A) of the first SC DNA oligonucleotide, wherein sequence (H) optionally has one or a plurality of abasic site(s) incorporated therein, and wherein at least a portion of the second signal DNA generation sequence (E) and sequence (G) that is complementary to the second signal DNA generation sequence (E) hybridize to form a hairpin structure. In these aspects and embodiments the methods, compositions, and kits can comprise a polymerase, and an endonuclease for a nicking reaction. In some embodiments, endonuclease recognition site (B) and/or (F) can be located in the loop of the hairpin structure. As discussed herein, any number, (n), of unique signal amplifier DNAs or cascade signal amplifier DNAs (or “cSA DNAs”) can be added to the reaction above, and may be structured such that each unique cSA DNA generates a unique Signal DNA sequence. For example, in some embodiments, n can be 10, 9, 8, 7, 5, 4, 3, 2, or 1, in which case 10, 9, 8, 7, 5, 4, 3, 2, or 1 different cSA DNAs are added to a reaction comprising a target nucleic and sequence conversion DNA (SC DNA). In embodiments of this aspect, the method also comprises determining the presence or absence of one or more signal DNA(s), wherein the presence of the one or more signal DNA(s) indicates the presence of the target nucleic acid in the sample.

For example, in some embodiments the disclosure relates to a method for detecting a target nucleic acid in a sample, said method comprising contacting said sample with: a first oligonucleotide (sequence conversion DNA or SC DNA) comprising, in the 5′ to 3′ direction, a first signal DNA generation sequence (A), an endonuclease recognition site (B), a complementary sequence that comprises a first complementary sequence (C) that is complementary to at least a portion of the signal DNA generation sequence (A), and a second complementary sequence (D) having at least one abasic site incorporated therein and that is complementary to the 3′ end of a target nucleic acid, wherein at least a portion of the first signal DNA generation sequence (A) and first complementary sequence (C) that is complementary to a portion of the signal DNA generation sequence (A) hybridize to form a hairpin structure. In further embodiments, the method comprises a second oligonucleotide (cascade signal amplifier DNA 1 or cSA DNA 1) comprising, in the 5′ to 3′ direction, a second unique signal DNA generation sequence (E), an endonuclease recognition site (F) (which may be the same or different from the endonuclease recognition site (B) in the SC DNA), a sequence region that includes a complementary sequence (G) that is complementary to at least a portion of the second unique signal DNA generation sequence (E), and a sequence (H) that is homologous to the first signal DNA generation sequence (A) of the first SC DNA oligonucleotide, wherein sequence (H) optionally has at least one abasic site incorporated therein, and wherein at least a portion of the second unique signal DNA generation sequence (E) and the sequence (G) that is complementary to the signal DNA generation sequence (E) hybridize to form a hairpin structure; a third oligonucleotide (cascade signal amplifier DNA 2 or cSA DNA 2) comprising, in the 5′ to 3′ direction, a third unique signal DNA generation sequence (I), an endonuclease recognition site (J) (which may be the same or different from the endonuclease recognition sites (B) and (F)), a sequence region that includes a complementary sequence (K) that is complementary to at least a portion of the third unique signal DNA generation sequence (I), and a sequence (L) that is homologous to the second signal DNA generation sequence (E) of the second oligonucleotide cSA DNA 1, wherein signal (L) optionally has at least one abasic site incorporated therein, and wherein at least a portion of the third unique signal DNA generation sequence (I) and the sequence (K) that is complementary to the signal DNA generation sequence (I) hybridize to form a hairpin structure. In such embodiments the method may further comprise a polymerase; and an endonuclease for a nicking reaction. In further embodiments, the method also comprises determining the presence or absence of a signal DNA, wherein the presence of the signal DNA indicates the presence of the target nucleic acid in the sample.

In another aspect, the disclosure relates to methods for detecting a target nucleic acid in a sample, wherein the interaction of a target nucleic acid with a first oligonucleotide (sequence conversion DNA or SC DNA having at least one abasic site) produces a first signal DNA (S1) that in turn interacts with a second oligonucleotide (cascade signal amplifier DNA 1 or cSA DNA 1, optionally having at least one abasic site) to produce a second signal DNA (S2) different from S1, which in turn can interact with a third oligonucleotide (cascade signal amplifier DNA 2 or cSA DNA 2, optionally having at least one abasic site) to produce Signal DNA (S1), which is the same Signal DNA generated upon interaction of the target nucleic acid with SC DNA. In this aspect, amplified Signal DNA (S2) is converted to Signal DNA (S1) upon interaction with cascade signal amplifier DNA cSA DNA 2, allowing cyclic amplification of signal DNA (S1).

In a still further aspect, the disclosure relates to methods for detecting a target nucleic acid in a sample, the methods comprising contacting said sample with: a first oligonucleotide (or sequence conversion DNA or SC DNA) comprising, in the 5′ to 3′ direction, a signal DNA generation sequence (A), an endonuclease recognition site (B), a complementary sequence that comprises a first complementary sequence (C) that is complementary to at least a portion of the signal DNA generation sequence (A), and a second complementary sequence (D) having at least one abasic site incorporated therein and that is complementary to the 3′ end of said target nucleic acid (T), wherein at least a portion of the first signal DNA generation sequence (A) and the first complementary sequence (C) that is complementary to the signal DNA generation sequence (A) hybridize to form a hairpin structure; a second oligonucleotide (or signal amplifier DNA or SA DNA) comprising, in the 5′ to 3′ direction, a signal DNA generation sequence (E) homologous to the signal DNA generation sequence (A) of the first oligonucleotide, an endonuclease recognition site (F) (which is the same as the endonuclease recognition site (B) of the first oligonucleotide), a sequence region that comprises a complementary sequence (G) that is complementary to at least a portion of the signal DNA generation sequence (E), and a sequence (H) that is homologous to the signal DNA generation sequence (A) of the first oligonucleotide, wherein sequence (H) optionally has at least one abasic site incorporated therein, wherein at least a portion of the second signal DNA generation sequence (E) and the sequence (G) that is complementary to the signal DNA generation sequence (E) hybridize to form a hairpin structure; a polymerase; and an endonuclease for a nicking reaction. In embodiments of this aspect, the method also comprises determining the presence or absence of a signal DNA, wherein the presence of the signal DNA indicates the presence of the target nucleic acid in the sample.

In certain embodiments one or more abasic sites (i.e., moieties) can be present in the sequence of any oligonucleotide described herein. For example, one or more abasic sites can be present in any signal conversion oligonucleotide (SC DNA), and/or in any signal amplification oligonucleotide (cSA DNA or SA DNA) prepared and/or used in accordance with the present disclosure. In certain embodiments there is at least one abasic site located at position 1 to 50, 1 to 45, 1 to 40, 1 to 35, 1 to 30, 1 to 25, 1 to 20, 1 to 15, 1 to 10, or 1 to 5 from the 3′-end of any SC DNA (e.g., within sequence (D)), and/or from the 3′-end of any signal amplification DNA (SA or cSA DNAs; e.g., within sequence (H) and/or sequence (L)). In the alternative, there is at least one abasic site located at position 1, 2, 3, 4, 5, 6, 7, 8, 9, 10, 11, 12, 13, 14, 15, 16, 17, 18, 19, 20, 21, 22, 23, 24, 25 or a combination thereof from the 3′-end of any SC DNA, and/or from the 3′-end of any cSA DNAs prepared and/or used in accordance with the present disclosure. In some embodiments, there is at least one abasic site located at position 1, 2, 3, 4, 5, 6, 7, 8, 9, 10, 11, 12, 13, 14, 15, 16, 17, 18, 19, 20, 21, 22, 23, 24, 25, or a combination thereof from the 5′-end of the sequence of any SC DNA complementary to the 3′-end of a target nucleic acid (e.g., sequence (D)), and/or the 5′-end of the sequence of any SA or cSA DNA complementary to the 3′-end of a signal DNA. In a further alternative, there is at least one abasic site located at position 1, 2, 3, 4, 5, 6, 7, 8, 9, 10, 11, 12, 13, 14, 15, 16, 17, 18, 19, 20, 21, 22, 23, 24, 25, or a combination thereof from the 3′-end of an endonuclease recognition site within any SC DNA, and/or any cSA DNA prepared or used according to the present invention.

As discussed further below, certain embodiments of the disclosure are illustrated that exemplify sequences that comprise one or more abasic sites in, for example, the SC DNA, are effective to reduce background signal in the performance of the methods disclosed herein (e.g., reducing signal that may result from target-independent amplification).

In some embodiments incorporating abasic sites in an SC DNA (within sequence (D) complementary to the 3′ end of a target nucleic acid), and/or in one or more cSA DNA(s) (e.g., sequence (L) that is homologous to the second signal DNA generation sequence (E)) can eliminate or reduce the generation of non-specific background signal in an amplification reaction. In other embodiments, incorporating abasic sites in the SC DNA and/or cSA DNA sequence can delay the generation/occurrence of non-specific amplification events for a period of time that allows for specific and accurate measurement of target nucleic acid. Thus, the disclosure provides for nucleic acid molecules (oligonucleotides), compositions, kits, and methods that allow for measurement of signal sequence(s) that indicate the presence of a target nucleic acid (e.g., a target sequence in a sample) before any detectable non-specific interfering background signal is generated.

As shown schematically in FIGS. 1A-1C (i.e., not shown to scale with regard to sequence length or number of hybridizing nucleotide pairs) the SC DNA and the cSA DNA comprise a sequence (C), (G), or (K) that is complementary to at least a portion of the signal DNA generation sequence (A), (E), or (I). At least a portion of sequence (C), (G), or (K) hybridizes to at least a portion of the complementary region of the signal DNA generation sequence (A), (E), or (I) and forms a hairpin (or stem loop) structure. As illustrated in the non-limiting embodiments of FIGS. 1A-1C, the double stranded stem is flanked on one side by an unpaired loop region comprising the endonuclease recognition site (B), (F), or (J), and on the other side by the sequence that is complementary to a target nucleic acid (in the case of SC DNA) or an upstream signal DNA generation sequence. In some embodiments the SC DNA can function as a cSA DNA (i.e., where the SC DNA both converts and amplifies signal from the target when the method comprises no separate amplifier oligonucleotide sequence).

In any signal conversion oligonucleotide (SC DNA), and/or in any signal amplification oligonucleotide (cSA DNA or SA DNA), the signal DNA generation sequence and the sequence that is complementary to at least a portion of the signal DNA generation sequence form a hairpin structure that, in some embodiments, can include the endonuclease recognition site, which comprises a sequence that is complementary to a sequence that is nicked by an endonuclease. The sequence that is nicked by the endonuclease may be within, downstream, or upstream from the sequence that is recognized by the endonuclease, and thus may form a double stranded sequence through such complementary hybridization. Suitably, when double stranded, the endonuclease recognition site (B), (F), or (J) can be recognized by an endonuclease present in the reaction, and the endonuclease recognition site (B), (F), or (J) (or a sequence adjacent to the endonuclease recognition site (B), (F), or (J)) may be cleaved on only one strand of the double-stranded DNA (i.e., nicked). In the case of SC DNA, binding of a target nucleic acid primes replication via DNA polymerase to create an active, double-stranded form of the endonuclease recognition site (B) that can serve as a recognition site for an endonuclease. Endonuclease nicking at the newly created double-stranded endonuclease site (B), or at a site adjacent to newly created double-stranded endonuclease site (B), then primes replication via DNA polymerase and generates signal DNA. For cSA DNA, binding of an upstream signal DNA primes replication via DNA polymerase to create an active, double-stranded form of the endonuclease recognition site (F) or (J) that can serve as a recognition site for an endonuclease. Endonuclease nicking at the newly created double-stranded endonuclease site (F) or (J), or at a site adjacent to newly created double-stranded endonuclease site (F) or (J), then primes replication via DNA polymerase and generates signal DNA. As illustrated in FIGS. 2A-2C, endonuclease recognition sites (B), (F), and (J) are oriented such that the newly replicated strand is nicked, not the SC DNA or cSA DNA. That is, when the newly replicated strand is generated the orientation of the endonuclease recognition site directs endonuclease activity (cleavage) of the newly replicated strand. As such, the endonuclease recognition site comprises a sequence that is complementary to a sequence that is nicked by an endonuclease, allowing the SC DNA oligonucleotide and the cSA DNA oligonucleotide to remain intact (i.e. is not nicked or cleaved) throughout the reaction.

The sequences (C), (G), and/or (K) is not limited by length, and can be from about 5 to about 100 nucleic acid bases, and all integers between 5 and 100. In some embodiments, the sequences (C), (G), and/or (K) are from about 5 to about 30 nucleic acid bases, and all integers between 5 and 30. Sequences (C), (G), and/or (K) are also not required to be the same length as the signal DNA generation sequences (A), (E), and/or (I). In some embodiments, (C), (G), and/or (K) can be the same length as the signal DNA generation sequences (A), (E), and/or (I), or it can be about 1-20 or about 1-10 bases shorter. The stem structure of a SC DNA and a cSA DNA may generally comprise a length of double stranded DNA ranging from about 3 to about 60 nucleic acid base pairs in length. In some embodiments, the stem comprises a length of double stranded DNA ranging from about 5 to about 20 nucleic acid base pairs, and all integers between 5 and 20.

The stem can also include bulges or mismatches, and sequences (C), (G), and/or (K) do not have to be 100% complementary to sequences (A), (E), and/or (I). In some embodiments, the sequences (C), (G), and/or (K) may be 100% complementary to all of, or to portions of, sequences (C), (G), and/or (K). For example, sequence (C) can be greater than about 50%, 60%, 70%, 80%, or 90% complementary to sequence (A), sequence (G) can be greater than about 50%, 60%, 70%, 80%, or 90% complementary to sequence (E), and sequence (K) can be greater than about 50%, 60%, 70%, 80%, or 90% complementary to sequence (I). Despite any base pairing mismatches, the two sequences generally have the ability to selectively hybridize to one another under appropriate conditions. In some embodiments, the amount of complementarity between sequence (A) and sequence (C), between sequence (E) and sequence (G), and between sequence (I) and sequence (K) is from about 80% to 100%, which can allow for hybridization under stringent or highly stringent conditions such as, for example, conditions disclosed herein.

In some embodiments, the double stranded stem of the stem-loop structure, for example, portions of sequences (A) and (C) of a SC DNA and portions of sequences (E) and (G) or sequences (I) and (K) can have a GC content that ranges from about 20% to about 70%, including any percentage between 20% and 70%.

In some embodiments, at least a portion of the endonuclease recognition site (B), (F), or (J) is located in the single stranded loop of the stem loop structure. In some embodiments, the entire endonuclease recognition site (B), (F), or (J) is located in the sequence comprising the loop region. Generally, the loop in the stem loop structure can be from about 3 to about 30 nucleotide bases and any number in between. In some embodiments, the endonuclease recognition site (B) is from 4 to about 10 nucleotide bases in length and all integers between 4 and 10.

The disclosure also provides for nucleic acid molecules (e.g., SC and cSA DNAs as disclosed herein), compositions, kits, and methods that allow for measurement of signal DNAs that indicate the presence of a target nucleic acid (e.g., a target sequence in a sample). For example, in some embodiments a signal resulting from the presence of from about 1 nM to about 1 fM target nucleic acid in a sample is detectable within about 10 to about 120, about 5 to about 120, or about 3 to about 120 minutes.

In embodiments of this aspect the polymerase may have strand displacement activity. In further embodiments, the polymerase may be 3′ to 5′ exonuclease deficient, 5′ to 3′ exonuclease deficient, or both 3′ to 5′ exonuclease deficient and 5′ to 3′ exonuclease deficient. In some embodiments the polymerase comprises a DNA polymerase.

In embodiments, the endonuclease may comprise a nicking endonuclease or a restriction endonuclease that can be used in a reaction that nicks an oligonucleotide. Endonuclease recognition sites (B) of the SC DNA, (E) of the first cSA DNA 1, (H) of the second cSA DNA 2, and/or any other endonuclease site located in any subsequent cSA DNAs can be identical, different, or a combination of the same and different endonuclease site(s) (e.g., wherein two are identical and a third is different).

While the method disclosed herein may be performed under typical DNA amplification conditions (e.g., typical temperatures associated with standard PCR, reactant concentrations, time cycles, etc.), in some embodiments, the method may be performed under isothermal conditions or under substantially constant temperatures. In further embodiments, the method may be performed at temperatures that are lower than temperatures used in standard PCR methods. As one example, some embodiments of the method may be performed at a temperature at or below a calculated optimal hybridization or annealing temperature, or an experimentally determined hybridization or annealing temperature, of the target nucleic acid (T) and the sequence (D) of the SC DNA, or of a signal DNA and the complementary sequence of a cSA DNA as described below. Some embodiments of the method can be performed at temperatures at or below the melting temperature of the hairpin structure in an oligonucleotide disclosed herein. In embodiments, the method may be performed at a temperature that is below the melting temperature of the target nucleic acid (T) bound to the sequence (D) of the SC DNA, or a signal DNA bound to the appropriate sequence of a cSA DNA. In yet other embodiments, the method may be performed at temperatures that allow for polymerase and/or endonuclease activity. In further embodiments, the method may be performed at temperatures that are at or about the optimal reaction temperature for the polymerase and/or endonuclease present in the reaction mixture for the detection of a target nucleic acid in a sample.

In another aspect, the disclosure relates to a chemically modified oligonucleotide, which may be referred herein as a “sequence conversion DNA” (or “SC DNA”) comprising, in the 5′ to 3′ direction, a first signal DNA generation sequence (A), an endonuclease recognition site (B), and a complementary sequence that comprises a first complementary sequence (C) that is complementary to at least a portion of the signal DNA generation sequence (A), and a second complementary sequence (D) having at least one abasic site incorporated therein and that is complementary to the 3′ end of a target nucleic acid, wherein at least a portion of the first signal DNA generation sequence (A) and the sequence (C) that is complementary to the signal DNA generation sequence (A) hybridize to form a hairpin structure.

In another aspect, the disclosure relates to oligonucleotides, which may be referred to herein as a “signal amplifier DNA”, “cascade signal amplifier DNA” (e.g., “SA DNA” or “cSA DNA”) which comprise, in the 5′ to 3′ direction, a unique signal DNA generation sequence (e.g., different from the signal DNA generation sequence (A) of a paired SC DNA), an endonuclease recognition site, and a sequence that comprises two sequence regions, wherein one sequence region is complementary to at least a portion of the unique signal DNA generation sequence, and the second sequence region has a sequence that optionally has at least one abasic site incorporated therein and that is homologous to a signal DNA generation sequence of a sequence conversion DNA (SC DNA) or to a signal DNA generation sequence of a different cSA DNA, wherein at least a portion of the unique signal DNA generation sequence and the sequence that is complementary to the unique signal DNA generation sequence hybridize to form a hairpin structure.

With reference to the Figures, for example, in another aspect the disclosure relates to an oligonucleotide, which may be referred to herein as a “first cascade signal amplifier DNA 1” (or “cSA DNA 1”) comprising, in the 5′ to 3′ direction, a second unique signal DNA generation sequence (E), an endonuclease recognition site (F), a sequence (G) that is complementary to at least a portion of the signal DNA generation sequence (E), and a sequence (H) that is homologous to a signal DNA generation sequence (A) of a sequence conversion DNA (SC DNA), wherein sequence (H) optionally has at least one abasic site incorporated therein, and wherein at least a portion of the second signal DNA generation sequence (E) and the sequence (G) that is complementary to the signal DNA generation sequence (E) hybridize to form a hairpin structure.

In a still further aspect, the disclosure relates to another oligonucleotide, which may be referred to herein as a “second cascade signal amplifier DNA 2” (or “cSA DNA 2”) comprising, in the 5′ to 3′ direction, a third unique signal DNA generation sequence (I), an endonuclease recognition site (J), a sequence (K) that is complementary to at least a portion of the signal DNA generation sequence (I), and a sequence (L) that is homologous to a signal DNA generation sequence (E) of a first cascade signal amplifier DNA 1 (cSA DNA 1), wherein sequence (L) optionally has at least one abasic site incorporated therein, and wherein at least a portion of the third signal DNA generation sequence (I) and the sequence (K) that is complementary to the signal DNA generation sequence (I) hybridize to form a hairpin structure.

The target nucleic acid sequence may be any nucleotide sequence of interest and in some embodiments may comprise a sequence that originates from an infectious agent or a microRNA. In other embodiments, the target nucleic acid may comprise a sequence from a gene that may be associated with a disease or a disorder.

In some embodiments, the endonuclease recognition sites in the oligonucleotides prepared and/or used in accordance with the present disclosure (e.g., SC DNA endonuclease recognition site (B), and cSA DNAs endonuclease recognition sites (E) and (H)) comprise a sequence that is complementary to a sequence that is nicked by an endonuclease. In other embodiments, the sequence that is nicked by the endonuclease is adjacent (downstream or upstream) to the sequence that is specifically recognized by the endonuclease.

In a further aspect, the disclosure relates to a composition for detecting a target nucleic acid in a sample, said composition comprising one or more of the oligonucleotides disclosed herein. Some example embodiments of this aspect provide a composition comprising: a first oligonucleotide (sequence conversion DNA or SC DNA) comprising, in the 5′ to 3′ direction, a first unique signal DNA generation sequence (A), an endonuclease recognition site (B), a complementary sequence that comprises a first complementary sequence (C) that is complementary to at least a portion of the signal DNA generation sequence (A), and a second complementary sequence (D) having at least one abasic site incorporated therein and that is complementary to the 3′ end of a target nucleic acid, wherein at least a portion of the first signal DNA generation sequence (A) and the sequence (C) that is complementary to the signal DNA generation sequence (A) hybridize to form a hairpin structure; a second oligonucleotide (cascade signal amplifier DNA 1 or cSA DNA 1) comprising, in the 5′ to 3′ direction, a second unique signal DNA generation sequence (E), an endonuclease recognition site (F), a complementary sequence comprising a sequence (G) that is complementary to at least a portion of the signal DNA generation sequence (E), and a sequence (H) that is homologous to the first signal DNA generation sequence (A) of the first SC DNA oligonucleotide, wherein sequence (H) optionally has at least one abasic site incorporated therein, and wherein at least a portion of the second signal DNA generation sequence (E) and the sequence (G) that is complementary to the signal DNA generation sequence (E) hybridize to form a hairpin structure; a third oligonucleotide (cascade signal amplifier DNA 2 or cSA DNA 2) comprising, in the 5′ to 3′ direction, a third unique signal DNA generation sequence (I), an endonuclease recognition site (J), a sequence region comprising a sequence (K) that is complementary to at least a portion of the signal DNA generation sequence (I), and a sequence (L) that is homologous to the second signal DNA generation sequence (E) of the second oligonucleotide cSA DNA 1, wherein sequence (L) optionally has at least one abasic site, and wherein signal (L) optionally has at least one abasic site incorporated therein, and wherein at least a portion of the third signal DNA generation sequence (I) and the sequence (K) that is complementary to the signal DNA generation sequence (I) hybridize to form a hairpin structure; a polymerase; and an endonuclease for a nicking reaction. In embodiments of this aspect, the compositions are used in methods for determining the presence or absence of a signal DNA, wherein the presence of the signal DNA indicates the presence of the target nucleic acid in the sample.

The compositions can also comprise a polymerase, and/or an endonuclease capable of nicking at or adjacent to the endonuclease recognition sites (endonuclease recognition site (B) of the SC DNA, and any endonuclease recognition sites of any cSA DNA present (e.g., endonuclease recognition site (F) of the first cSA DNA 1, and endonuclease recognition site (J) of the second cSA DNA 2)), when the endonuclease recognition sites are made double stranded. In each covered structure (i.e., hairpin structure), when the newly replicated strand is generated the orientation of the endonuclease recognition site directs endonuclease activity (i.e., nicking/cleavage) of the newly replicated strand. As such, the endonuclease recognition site comprises a sequence that is complementary to a sequence that is nicked by an endonuclease, allowing the SC DNA or cSA DNA to remain intact throughout the reaction. Compositions can also include other reagents such as reaction buffers, deoxyribonucleotides, and reporter molecules such as, for example, fluorophore-modified probe DNAs (e.g., molecular beacon probes) for the fluorescent detection of newly synthesized DNA.

In yet another aspect, the disclosure relates to a kit for detecting a target nucleic acid in a sample, said kit comprising: a first oligonucleotide (sequence conversion DNA or SC DNA) comprising, in the 5′ to 3′ direction, a first signal DNA generation sequence (A), an endonuclease recognition site (B), a complementary sequence that comprises a first complementary sequence (C) that is complementary to at least a portion of the signal DNA generation sequence (A), and a second complementary sequence (D) having at least one abasic site incorporated therein and that is complementary to the 3′ end of a target nucleic acid, wherein at least a portion of the first signal DNA generation sequence (A) and the sequence (C) that is complementary to the signal DNA generation sequence (A) hybridize to form a hairpin structure; a second oligonucleotide (cascade signal amplifier DNA 1 or cSA DNA 1) comprising, in the 5′ to 3′ direction, a second unique signal DNA generation sequence (E), an endonuclease recognition site (F), a sequence region comprising a sequence (G) that is complementary to at least a portion of the signal DNA generation sequence (E), and a sequence (H) that is homologous to the first signal DNA generation sequence (A) of the first SC DNA oligonucleotide, wherein sequence (H) optionally has at least one abasic site incorporated therein, and wherein sequence (H) optionally has at least one abasic site incorporated therein, and wherein at least a portion of the second signal DNA generation sequence (E) and the sequence (G) that is complementary to the signal DNA generation sequence (E) hybridize to form a hairpin structure; and a third oligonucleotide (cascade signal amplifier DNA 2 or cSA DNA 2) comprising, in the 5′ to 3′ direction, a third unique signal DNA generation sequence (I), an endonuclease recognition site (J), a sequence region comprising a sequence (K) that is complementary to at least a portion of the signal DNA generation sequence (I), and a sequence (L) that is homologous to the second signal DNA generation sequence (E) of the second oligonucleotide cSA DNA 1, wherein sequence (L) optionally has at least one abasic site incorporated therein, and wherein signal (L) optionally has at least one abasic site incorporated therein, and wherein at least a portion of the third signal DNA generation sequence (I) and the sequence (K) that is complementary to the signal DNA generation sequence (I) hybridize to form a hairpin structure. In some embodiments the kits can further comprise a polymerase and/or an endonuclease capable of nicking an endonuclease recognition site or a site adjacent to an endonuclease recognition site. The kits can also include reagents such as reaction buffers, deoxyribonucleotides, and reporter molecules such as, for example, fluorophore-modified probe DNAs (e.g., molecular beacon probes) for the fluorescent detection of newly synthesized DNA such as a signal DNA. The kits can also comprise instructions for use in the practice of any one of the methods disclosed herein.

The methods, oligonucleotides, compositions, and kits disclosed herein may be used in combination with integrated system platforms. For example, methods, oligonucleotides, compositions, and kits of the present invention may be used in combination systems commercially marketed, e.g., by Abbott Laboratories (Abbott Park, IL) as, for example, ARCHITECT® or the series of Abbott Alinity devices. The methods, oligonucleotides, compositions, and kits disclosed herein may be used with sample preparation system platforms such as, for example, the m2000sp sample preparation system (Abbott Diagnostics, Abbott Park, IL). Similarly, the methods, oligonucleotides, compositions, and kits disclosed herein may be used with point-of-care system platforms such as, for example, Abbott's i-STAT point-of-care system (e.g., i-STAT and i-STAT Alinity, Abbott Diagnostics, Abbott Park, IL), and optionally may be employed on other platforms (e.g., Universal Biosensors (Rowville, Australia) (see, e.g., US 2006/0134713), Axis-Shield PoC AS (Oslo, Norway) and Clinical Lab Products (Los Angeles, USA)). Further, the methods, oligonucleotides, compositions, and kits of the present invention can be used with any number of other devices, assay platforms, and instrumentation such as, for example, hand held fluorescence detectors, micro-pH meters, microfluidic devices, microarrays, enzymatic detection systems, immunochromatographic strips, and lateral flow devices.

The methods, oligonucleotides, compositions, and kits disclosed herein may be used in the field of molecular diagnostics, including diagnosis of non-infectious and infectious diseases. For example, methods, oligonucleotides, compositions, and kits of the present invention can be used to detect cancers and other genetic diseases. Similarly, methods, oligonucleotides, compositions, and kits of the present invention can be used to detect target nucleic acids originating from infectious diseases such as, for example, hepatitis B virus, hepatitis C virus, human immunodeficiency virus, Chlamydia trachomatis, Neisseria gonorrhoeae, influenza A virus, influenza B virus, or respiratory syncytial virus.

Additional aspects, embodiments, and advantages provided by the disclosure will become apparent in view of the description that follows.

### BRIEF DESCRIPTION OF THE DRAWINGS

FIG. 1A is a diagram schematically illustrating (i.e., not to scale) a non-limiting example of a Sequence Conversion DNA (SC DNA) for the detection of a target nucleic acid in a sample. The SC DNA comprises, in the 5′ to 3′ direction, a first signal generation sequence (A), an endonuclease recognition site (B) that can be used in a nicking reaction, and a complementary sequence comprising a first complementary sequence (C) that is complementary to at least a portion of the signal DNA generation sequence (A), and a second complementary sequence (D) having at least one abasic site incorporated therein and that is complementary to the target nucleic acid, wherein at least a portion of the first signal DNA generation sequence (A) and the sequence (C) that is complementary to the signal DNA generation sequence (A) hybridize to form a hairpin structure.

FIG. 1B is a diagram schematically illustrating (i.e., not to scale) a non-limiting example of a first cascade Signal Amplifier DNA 1 (cSA DNA 1) for the detection of a target nucleic acid in a sample. The cSA DNA 1 comprises, in the 5′ to 3′ direction, a second unique signal DNA generation sequence (E); an endonuclease recognition site (F), and a sequence region comprising a sequence (G) that is complementary to at least a portion of the signal DNA generation sequence (E), and a sequence (H) that is homologous to the first signal DNA generation sequence (A) of a first SC DNA, wherein sequence (H) optionally has at least one abasic site incorporated therein, and wherein at least a portion of the second signal DNA generation sequence (E) and the sequence (G) that is complementary to the signal DNA generation sequence (E) hybridize to form a hairpin structure.

FIG. 1C is a diagram schematically illustrating (i.e., not to scale) a non-limiting example of a second cascade Signal Amplifier DNA 2 (cSA DNA 2) for the detection of a target nucleic acid in a sample. The cSA DNA 2 comprises, in the 5′ to 3′ direction, a third signal DNA generation sequence (I); an endonuclease recognition site (J), and a sequence region comprising a sequence (K) that is complementary to at least a portion of the signal DNA generation sequence (I), and a sequence (L) that is homologous to the second signal DNA generation sequence (E) of a first cSA DNA 1, wherein sequence (L) optionally has at least one abasic site incorporated therein, and wherein at least a portion of the third signal DNA generation sequence (I) and the sequence (K) that is complementary to the signal DNA generation sequence (I) hybridize to form a hairpin structure.

FIG. 2A is a diagram schematically illustrating (i.e., not to scale) the progression of an exemplary reaction of a target (T) nucleic acid with a Sequence Conversion (SC) DNA of the present disclosure for the detection of a target nucleic acid in a sample. Sequences (A)-(D) are as described in FIG. 1A, sequence (T) represents a target sequence, sequence (X) represents the sequence produced when Target (T) bound to sequence (D) is extended by polymerase, sequence (X′) represents the nicked extension sequence, and sequence (S1) represents the first signal DNA sequence eventually produced upon binding of the target (T) nucleic acid to the SC DNA.

FIG. 2B is a diagram schematically illustrating (i.e., not to scale) the progression of an exemplary reaction of a signal DNA (S1) with a first cascade Signal Amplifier DNA 1 (cSA DNA 1) for the detection of a target nucleic acid in a sample. Sequences (E)-(H) are as described in FIG. 1B, sequence (S1) is the Signal DNA produced from reaction of Target (T) nucleic acid with SC DNA as described in FIG. 2A, sequence (Y) represents the sequence produced when Signal DNA (S1) bound to sequence (H) is extended by polymerase, sequence (Y′) represents the nicked extension sequence, and sequence (S2) represents the unique signal DNA sequence eventually produced. Because the cSA DNA 1 signal generation sequence (E is non-homologous to the SC signal generation sequence (A), a different unique signal DNA (S2) is produced.

FIG. 2C is a diagram schematically illustrating (i.e., not to scale) the progression of an exemplary reaction of a signal DNA (S2) with a second cascade Signal Amplifier DNA 2 (cSA DNA 2) for the detection of a target nucleic acid in a sample. Sequences (I)-(L) are as described in FIG. 1C, signal sequence (S2) is the Signal DNA produced as described in FIG. 2B, sequence (U) represents the sequence produced when Signal DNA (S2) bound to sequence (L) is extended by polymerase, sequence (U′) represents the nicked extension sequence, and sequence (S3) represents the unique signal DNA sequence eventually produced. Because the cSA DNA 2 signal generation sequence (I) is non-homologous to the cSA 1 signal generation sequence (E), a different unique signal DNA (S3) is produced.

FIG. 3A depicts the results of reactions performed in Example 1 of this disclosure.

FIG. 3B depicts the results of reactions performed in Example 1 of this disclosure.

FIG. 4 depicts the results of reactions performed in Example 2 of this disclosure.

FIG. 5 depicts the results of reactions performed in Example 3 of this disclosure.

FIG. 6 depicts the results of reactions performed in Example 4 of this disclosure.

FIG. 7 depicts the results of reactions performed in Example 5 of this disclosure.

FIG. 8 depicts the results of reactions performed in Example 6 of this disclosure.

FIG. 9 depicts the results of reactions performed in Example 7 of this disclosure.

FIG. 10 depicts the results of reactions performed in Example 8 of this disclosure.

FIG. 11 depicts the results of reactions performed in Example 9 of this disclosure.

FIG. 12 depicts the results of reactions performed in Example 10 of this disclosure.

FIG. 13 depicts the results of reactions performed in Example 11 of this disclosure.

FIG. 14 depicts the results of reactions performed in Example 12 of this disclosure.

FIG. 15 depicts the results of reactions performed in Example 13 of this disclosure.

FIG. 16 depicts the results of reactions performed in Example 14 of this disclosure.

FIG. 17 depicts the results of reactions performed in Example 14 of this disclosure.

FIG. 18 depicts the results of reactions performed in Example 14 of this disclosure.

FIG. 19A depicts the results of reactions performed in Example 15 of this disclosure.

FIG. 19B depicts the results of reactions performed in Example 15 of this disclosure.

FIG. 19C depicts the results of reactions performed in Example 15 of this disclosure.

### DETAILED DESCRIPTION

In a general sense, the disclosure relates to nucleic acid constructs that are surprisingly effective in the detection of target nucleic acids in a test sample. The constructs disclosed herein comprise nucleic acid sequences that allow the production of signal DNAs that are generated in the presence of a target nucleic acid. The methods and nucleic acid constructs disclosed herein provide for selective and sensitive detection of target nucleic acids that may be advantageously performed under low temperature and isothermal conditions.

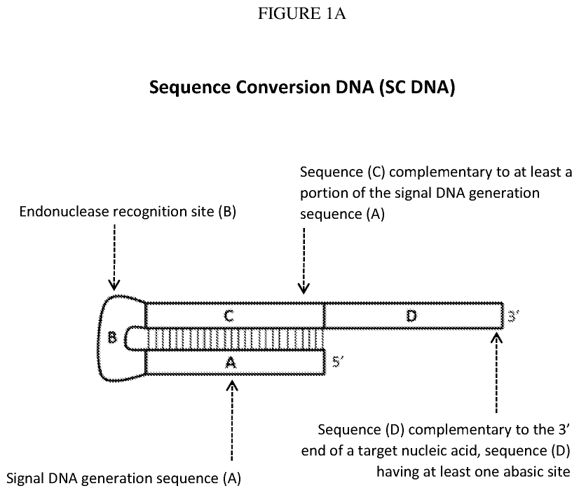

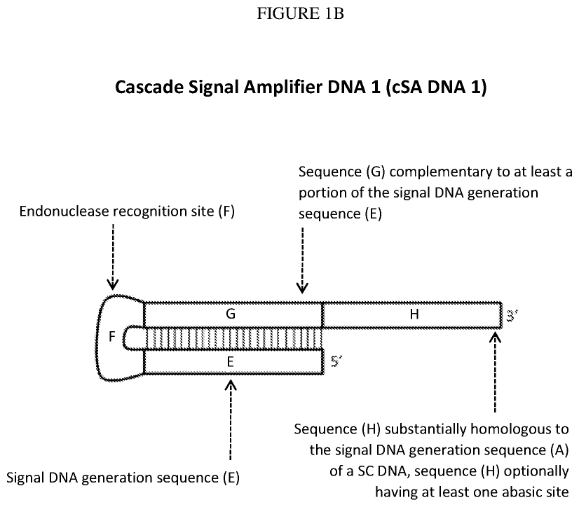

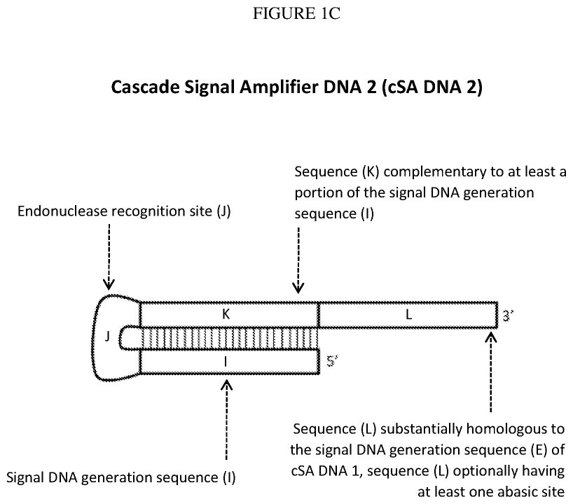

In embodiments of this aspect, the disclosure provides novel Sequence Conversion (SC) and cascade Signal Amplifier (cSA) oligonucleotide constructs, and combinations thereof, that are useful in detecting a target nucleic acid in a sample. As depicted by the illustrative embodiment of FIG. 1A, a Sequence Conversion DNA (SC DNA) oligonucleotide for the detection of a target nucleic acid in a sample comprises, in the 5′ to 3′ direction, a first signal generation sequence (A), an endonuclease recognition site (B) that can be used in a nicking reaction, and a complementary sequence that comprises at least one abasic moiety and wherein the complementary sequence comprises a first complementary sequence (C) that is complementary to at least a portion of the signal DNA generation sequence (A), and a second complementary sequence (D) having the at least one abasic site incorporated therein and that is complementary to the target nucleic acid, wherein at least a portion of the first signal DNA generation sequence (A) and the sequence (C) that is complementary to the signal DNA generation sequence (A) hybridize to form a hairpin structure. In some embodiments of this aspect, the complementary sequence (i.e., comprising (C) and (D)) comprises a plurality of abasic moieties. In some further embodiments, the second complementary sequence (D), which is complementary to the 3′ end of a target nucleic acid, comprises a plurality of abasic sites (e.g., 2, 3, 4, 5, 6, 7, 8, 9, or 10 abasic sites). In some embodiments, the chemically modified SC DNA oligonucleotide further comprises a 3′-end modification.

As depicted by the illustrative embodiment of FIG. 1B, a first cascade Signal Amplifier DNA (cSA DNA 1) for the detection of a target nucleic acid in a sample comprises, in the 5′ to 3′ direction, a second unique signal DNA generation sequence (E); an endonuclease recognition site (F) (that can be the same as or different from endonuclease recognition site (B)), a sequence region comprising a sequence (G) that is complementary to at least a portion of the signal DNA generation sequence (E), and a sequence (H) that is homologous to the first signal DNA generation sequence (A) of a first SC DNA, wherein sequence (H) optionally has at least one abasic site incorporated therein, and wherein at least a portion of the second signal DNA generation sequence (E) and the sequence (G) that is complementary to the signal DNA generation sequence (E) hybridize to form a hairpin structure. In some embodiments of this aspect, the sequence (H), which is homologous to the first signal DNA generation sequence (A) of a first SC DNA, comprises a plurality of abasic sites (e.g., 2, 3, 4, 5, 6, 7, 8, 9, or 10 abasic sites). In some embodiments, the chemically modified oligonucleotide further comprises a 3′-end modification.

As depicted by the illustrative embodiment of FIG. 1C, a second cascade Signal Amplifier DNA 2 (cSA DNA 2) for the detection of a target nucleic acid in a sample comprises, in the 5′ to 3′ direction, a third unique signal DNA generation sequence (I); an endonuclease recognition site (J) (that can be the same as or different from endonuclease recognition sites (B) and (F)), a sequence region comprising a sequence (K) that is complementary to at least a portion of the signal DNA generation sequence (I), and a sequence (L) that is homologous to the second signal DNA generation sequence (E) of a first cSA DNA 1, wherein sequence (L) optionally has at least one abasic site incorporated therein, and wherein at least a portion of the third signal DNA generation sequence (I) and the sequence (K) that is complementary to the signal DNA generation sequence (I) hybridize to form a hairpin structure. In some embodiments of this aspect, the sequence (L), which is homologous to the second signal DNA generation sequence (E) of a first cSA DNA 1, comprises a plurality of abasic sites (e.g., 2, 3, 4, 5, 6, 7, 8, 9, or 10 or more abasic sites). In some embodiments, the chemically modified oligonucleotide further comprises a 3′-end modification.

In embodiments of the present disclosure, sequence (D) of the SC DNA has at least one abasic site incorporated therein and is complementary to a target nucleic acid, and the sequence of a cSA DNA substantially homologous to the signal DNA generation sequence of a SC DNA or of a different cSA DNA optionally has at least one abasic site incorporated therein. In other embodiments, only sequence (D) of the SC DNA comprises one or more abasic sites incorporated therein. The abasic site has the following general structure:

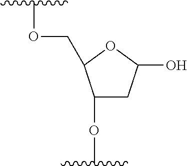 [SMILES: *OCC1OC(O)CC1O*]

As illustrated, an abasic site, also known as an apurinic/apyrimidinic site (AP site), is a location in DNA (and also in RNA) that has neither a purine nor a pyrimidine base.

By incorporating abasic sites into, for example, the sequence (D) of a SC DNA and/or optionally into the sequence of a cSA DNA substantially homologous to the signal DNA generation sequence of a SC DNA or of a different cSA DNA described herein, non-specific background signal amplification is either eliminated entirely, reduced, or delayed for a period of time that is adequate to detect signal sequence resulting from the presence of target nucleic acid without any substantial interference from non-specific background signal. For example, in certain embodiments there is between a 99 to 10% decrease, a 99 to 20% decrease, a 99 to 30% decrease, a 99 to 50% decrease, or a 99% to 70% decrease in non-specific background signal amplification when using methods, oligonucleotides, compositions, and kits of the present invention. In certain embodiments, incorporating one or more abasic sites into the sequence (D) of a SC DNA and into the portion of the sequence of a SA DNA that is substantially homologous to the signal DNA generation sequence of a SC DNA, can provide reduced non-specific background signal amplification by an average of at least about 10%, 20%, 30%, 40%, or 50% or more, or by an average of about 10%, about 20%, or about 30%.

As illustrated in the Figures herein, the SC and cSA DNAs disclosed herein comprise signal generation sequences (e.g., signal DNA generation sequence (A) of the SC DNA; signal generation sequence (E) of cSA DNA 1; and signal DNA generation sequence (I) of cSA DNA 2). These signal generation sequences can comprise any desired nucleic acid sequence and are not limited by any particular sequence. As discussed in greater detail below, these signal generation sequences provide at least a portion of the template for the generation of signal DNA (e.g., S1, S2, and S3). These signal generation sequences are not limited by length. In some embodiments, the signal generation sequence in the SC DNA and/or cSA DNA(s) of the present disclosure are from about 5 to about 100 nucleic acid bases, and all integers between 5 and 100. In some embodiments, the signal generation sequence in the SC DNA and/or cSA DNA(s) are from about 5 to about 30 nucleic acid bases, and all integers between 5 and 30. In some embodiments, the signal generation sequence in the SC DNA and/or cSA DNA(s) are from about 10 to about 30 nucleic acid bases, and all integers between 10 and 30. In yet further embodiments, the signal generation sequence in the SC DNA and/or cSA DNA(s) are from about 15 to about 30 nucleic acid bases, and all integers between 15 and 30 (e.g., about 16, about 17, about 18, about 19, about 20, about 21, about 22, about 23, about 24, about 25, about 26, about 27, about 28, about 29 or about 30 bases).

In a further aspect, the lengths of the: signal DNA generation sequence (A); sequence (D) having at least one abasic site incorporated therein and that is complementary to the 3′ end of a target nucleic acid; signal DNA generation sequence (E); and the sequence (H) that is substantially homologous to the signal DNA generation sequence (A) may vary, but typically each of these sequences is about the same length as the other. In some embodiments, the length of one or a combination of these sequences may be in a range from about 5 to about 100 nucleotides, but are more typically from about 5 to about 30, from about 10 to about 30, or from about 15 to about 30 nucleotides in length. The endonuclease recognition site comprises a sequence that can be recognized, bound, and nicked by an endonuclease as described herein. Such sequences are generally known in the art. The endonuclease recognition site can comprise additional nucleotides either 5′ or 3′ to the endonuclease binding site (or both 5′ and 3′), but is typically no more than 10 nucleotides in length.

In certain embodiments described herein, the location of the abasic site, or the plurality of abasic sites, in the sequence (D) of the SC DNA complementary to the 3′-end of a target nucleic acid, and optionally in the sequence of any cSA DNA substantially homologous to the signal DNA generation sequence of a SC DNA or of a different cSA DNA (e.g., the sequence (H) of the SA DNA substantially homologous to the signal DNA generation sequence (A) of the SC DNA) are identified with respect to the 3′-end of the sequence, and may vary. In some embodiments the location of the abasic site or the plurality of abasic sites in the sequence may be identified with respect to the 5′-end of the sequence that is complementary to the target nucleic acid, the 5′-end of the sequence that is complementary to a signal sequence, or the 3′-end of the endonuclease recognition site. Thus, in some embodiments the abasic site, or sites, is located at position 3, 4, 5, 6, 7, 8, 9, 10, 11, 12, 13, 14, 15 or a combination thereof from the 5′ end of the sequence complementary to the 3′ end of the target nucleic acid (or, alternatively, at those same positions relative to the location of the 3′ end of the endonuclease recognition site). In some embodiments, the abasic site is located at position 7 from the 5′ side of said sequence complementary to the 3′ end of said target nucleic acid. In some embodiments, two abasic sites are located at positions 7 and 8 from the 5′ side of said sequence complementary to the 3′ end of said target nucleic acid. In some embodiments, three abasic sites located at positions 7, 8, and 9 from the 5′ side of said sequence complementary to the 3′ end of said target nucleic acid. In further embodiments, four abasic sites located at positions 7, 8, 9, and 10 from the 5′ side of said sequence complementary to the 3′ end of said target nucleic acid.

In some alternative embodiments, the abasic site or the plurality of abasic sites is located at position(s) 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 from the 3′-end of the oligonucleotide sequence. In some embodiments that comprise a single abasic site, the abasic site is located at any of positions 1, 4, 5, 6, 7, 8, 9, 10, 11, 12, 13, 14, 15, 16, 17, 18, 19, 20, 21, 22, 23, 24, 25, 26, 27, 28, 29, or 30 from the 3′-end of the oligonucleotide sequence.

In embodiments wherein the sequence (D) of the SC DNA complementary to the 3′-end of a target nucleic acid, and/or the sequence of any cSA DNA substantially homologous to the signal DNA generation sequence of a SC DNA or of a different cSA DNA comprise two abasic sites, the abasic sites may be located at any combination of two positions selected from positions 1 to 30 from the 3′-end of the respective sequence.

In embodiments wherein the sequence (D) of the SC DNA complementary to the 3′-end of a target nucleic acid, and/or the sequence of any cSA DNA substantially homologous to the signal DNA generation sequence of a SC DNA or of a different cSA DNA comprise three abasic sites, the abasic sites may be located at any combination of three positions selected from positions 1 to 30 from the 3′-end of the respective sequence.

In embodiments wherein the sequence (D) of the SC DNA complementary to the 3′-end of a target nucleic acid, and/or the sequence of any cSA DNA substantially homologous to the signal DNA generation sequence of a SC DNA or of a different cSA DNA comprise four abasic sites, the abasic sites may be located at any combination of four positions selected from positions 1 to 30 from the 3′-end of the respective sequence.

The SC DNA and cSA DNAs comprise endonuclease recognition sites (e.g., SC DNA endonuclease site (B), and cSA DNA 1 endonuclease site (F), and cSA DNA 2 endonuclease site (J)), which can be the same or different. In single stranded form (e.g., the structure of FIGS. 1A, 1B, and 1C) the endonuclease recognition sites (e.g., endonuclease recognition sites (B), (F), and (J)) comprise a sequence that is complementary to a sequence that is nicked by an endonuclease. The sequence that is nicked by the endonuclease may be within, downstream, or upstream from the sequence that is recognized by the endonuclease. Suitably, when double stranded, the endonuclease recognition sites (e.g. endonuclease recognition sites (B), (F), and (J)) can be recognized by one or more endonucleases present in the reaction, and the endonuclease recognition sites (or a sequence adjacent to the endonuclease recognition sites) may be cleaved on only one strand of the double-stranded DNA (i.e., nicked).

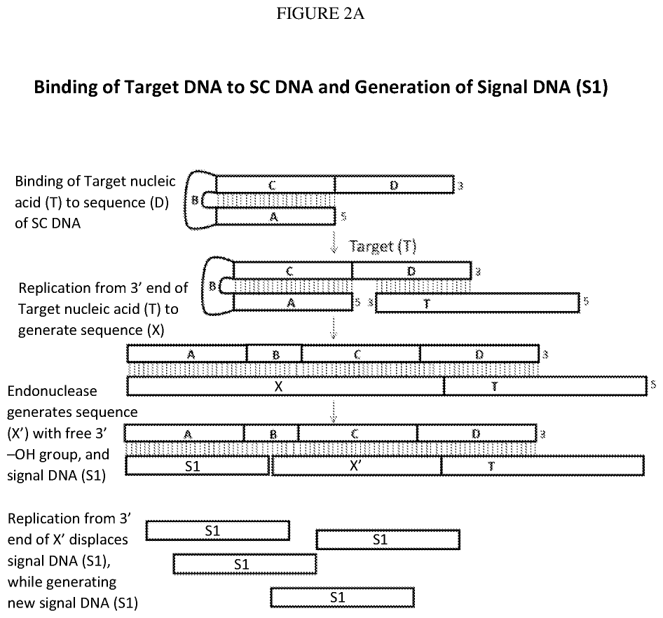

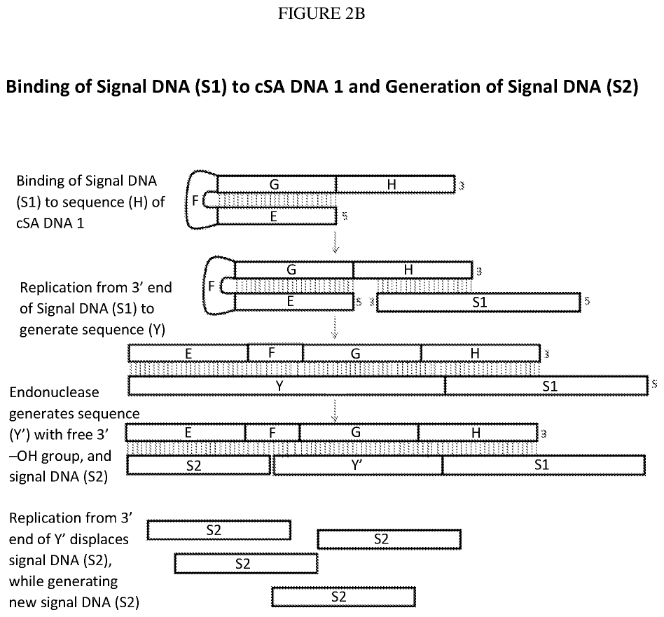

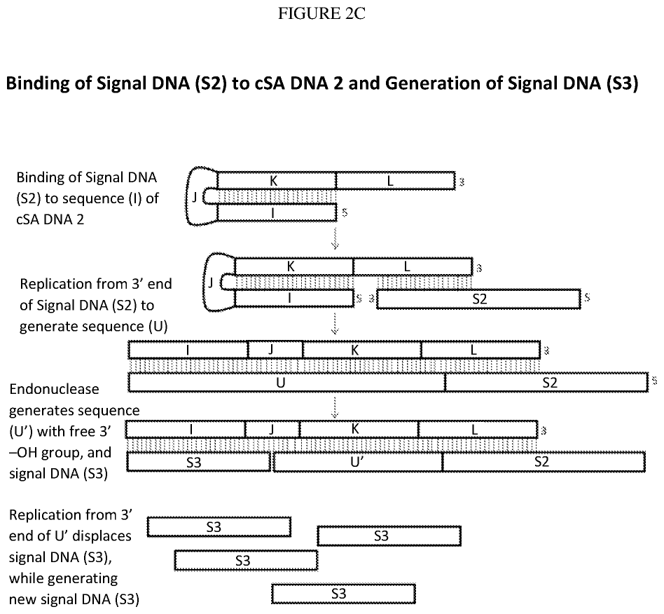

As described in greater detail below, binding of a target nucleic acid to the complementary sequence (D) of the SC DNA primes replication via DNA polymerase to create an active, double-stranded form of the endonuclease recognition site (B) that can then serve as a recognition site for an endonuclease (FIG. 2A). Endonuclease nicking at the newly created double-stranded endonuclease site (B), or at a site adjacent to newly created double-stranded endonuclease site (B), then primes replication via DNA polymerase and generates a first signal DNA (S1) (see, e.g., FIG. 2A). As illustrated in FIG. 2A, the endonuclease recognition site (B) is oriented such that the newly replicated strand is nicked, not the SC DNA. That is, when the newly replicated strand is generated, the orientation of the endonuclease recognition site in (B) directs endonuclease activity (cleavage) of the newly replicated strand. As such, the endonuclease recognition site comprises a sequence that is complementary to a sequence that is nicked by an endonuclease, allowing the SC oligonucleotide to remain intact throughout the reaction (i.e., the SC DNA is not nicked or cleaved).

As described in greater detail below, binding of a first signal DNA (S1), generated from the signal generation sequence (A) of a SC DNA, to the sequence (H) of a cSA DNA 1 primes replication via DNA polymerase to create an active, double-stranded form of the endonuclease recognition site (F) of the cSA DNA 1 that can serve as a recognition site for an endonuclease (FIG. 2B). Endonuclease nicking at the newly created double-stranded endonuclease site (F) of the cSA DNA 1, or at a site adjacent to newly created double-stranded endonuclease site (F), then primes replication via DNA polymerase and generates a second signal DNA (S2) that is different from the first signal DNA (S1) generated from the SC DNA (FIG. 2B). As illustrated in FIG. 2B, the endonuclease recognition site (F) is oriented such that the newly replicated strand is nicked, not the cSA DNA 1. That is, when the newly replicated strand is generated the orientation of the endonuclease recognition site in (F) directs endonuclease activity (cleavage) of the newly replicated strand. As such, the endonuclease recognition site comprises a sequence that is complementary to a sequence that is nicked by an endonuclease, allowing the cSA DNA 1 oligonucleotide to remain intact throughout the reaction (i.e., the cSA DNA 1 is not nicked or cleaved).

As described in greater detail below, binding of a second signal DNA (S2), generated from the signal generation sequence (E) of a first cSA DNA 1, to the sequence (L) of a cSA DNA 2 primes replication via DNA polymerase to create an active, double-stranded form of the endonuclease recognition site (J) of the cSA DNA 2 that can serve as a recognition site for an endonuclease (FIG. 2C). Endonuclease nicking at the newly created double-stranded endonuclease site (J) of the cSA DNA 2, or at a site adjacent to newly created double-stranded endonuclease site (J), then primes replication via DNA polymerase and generates a third signal DNA (S3) that is different from the first signal DNA (S1) generated from the SC DNA (FIG. 2A), and from the second signal DNA (S2) generated from the cSA DNA 1 (FIG. 2B). As illustrated in FIG. 2C, the endonuclease recognition site (J) is oriented such that the newly replicated strand is nicked, not the cSA DNA 2. That is, when the newly replicated strand is generated the orientation of the endonuclease recognition site in (J) directs endonuclease activity (cleavage) of the newly replicated strand. As such, the endonuclease recognition site comprises a sequence that is complementary to a sequence that is nicked by an endonuclease, allowing the cSA DNA 2 oligonucleotide to remain intact throughout the reaction (i.e., the cSA DNA 2 is not nicked or cleaved).

The sequence (D) of the SC DNA that is complementary to the target DNA and that has at least one abasic site is not limited by length, and can be from about 5 to about 100 nucleic acid bases, and all integers between 5 and 100. In some embodiments, the sequence (D) of the SC DNA is from about 5 to about 30 nucleic acid bases, and all integers between 5 and 30. In some embodiments, the sequence (D) in the SC DNA is from about 10 to about 30 nucleic acid bases, and all integers between 10 and 30. In further embodiments, the sequence (D) of the SC DNA is from about 15 to about 30 nucleic acid bases, and all integers between 15 and 30.

Complementary sequences are capable of forming hydrogen bonding interactions to form a double stranded nucleic acid structure (e.g., nucleic acid base pairs). For example, a sequence that is complementary to a first sequence includes a sequence which is capable of forming Watson-Crick base-pairs with the first sequence. As used herein, the term “complementary” does not require that a sequence is complementary over the full-length of its complementary strand, and encompasses a sequence that is complementary to a portion of another sequence. Thus, in some embodiments, a complementary sequence encompasses sequences that are complementary over the entire length of the sequence or over a portion thereof (e.g., greater than about 5%, about 10%, about 20%, about 30%, about 40%, about 50%, about 60%, about 70%, about 80%, about 90%, or about 95% of the length of the sequence). For example, two sequences can be complementary to each other over a length ranging from about 2 to about 100 consecutive (contiguous) nucleotides, or any integer between 2 and 100. In some embodiments, two sequences can be complementary to each other over a length ranging from about 15 to about 30 consecutive (contiguous) nucleotides, or any integer between 15 and 30. As used herein, complementary sequences can encompass sequences that have some sequence mismatches. For example, complementary sequences can include sequences that are complementary to at least about 70% to 100%, preferably greater than above 95% of the length of the sequence. Despite some amount of mismatches, complementary sequences generally have the ability to selectively hybridize to one another under appropriate conditions such as, for example, stringent and highly stringent conditions such as those described herein or generally known by those of ordinary skill in the art.

The SC and cSA DNAs may be synthesized by known methods. For example, the SC and cSA DNAs can be synthesized using a phosphoramidite method, a phosphotriester method, an H-phosphonate method, or a thiophosphonate method. In some embodiments, the SC and/or cSA DNAs can be purified, for example using ion exchange HPLC.

The SC and cSA DNAs may comprise chemical modifications (in addition to the incorporation of abasic groups) such as are generally known in the art. In some embodiments, for example, the SC and cSA DNAs can comprise chemically modified nucleotides (e.g., 2′-O methyl derivative, phosphorothioates, etc.), 3′ end modifications, 5′ end modifications, or any combinations thereof. In some embodiments, the 3′ end of the SC and cSA DNAs may be modified such that an extension reaction does not occur from the 3′ end of the SC or cSA DNA (e.g., upon binding of a target sequence, or another non-target sequence, that might serve as a primer for polymerase extension). As illustrated in FIG. 2A, it is the 3′ end of the target nucleic acid (T), not the SC DNA, which initiates DNA replication. Any replication initiated from the 3′end of the SC or cSA DNAs may lead to detection errors (e.g., false positives). Further, non-specific extension reactions from an unmodified 3′ end of the SC DNA arising from events such as, for example, binding between the SC DNA and a non-target sequence, binding between the SC DNA and a target sequence at an incorrect position, binding between SC and cSA DNAs, or non-templated de novo or ab initio DNA synthesis may also lead to detection errors. Accordingly, in embodiments, the SC and cSA DNAs comprise a 3′ end modification that can reduce or eliminate the occurrence of any non-desired extension reactions, such as those discussed above. Non-limiting examples of 3′-end modifications include TAMRA, DABCYL, and FAM. Other non-limiting examples of modifications include, for example, biotinylation, fluorochromation, phosphorylation, thiolation, amination, inverted nucleotides, or abasic groups.

In another aspect, the present invention encompasses methods for detecting a target nucleic acid (T) in a sample. The methods generally comprise contacting said sample with: a first oligonucleotide (sequence conversion DNA or SC DNA) comprising, in the 5′ to 3′ direction, a first signal DNA generation sequence (A), an endonuclease recognition site (B), and a complementary sequence that comprises at least one abasic moiety, a first complementary sequence (C) that is complementary to at least a portion of the signal DNA generation sequence (A), and a second complementary sequence (D) having the at least one abasic site incorporated therein and that is complementary to the 3′ end of a target nucleic acid, wherein at least a portion of the first signal DNA generation sequence (A) and the sequence (C) that is complementary to the signal DNA generation sequence (A) hybridize to form a hairpin structure; a second oligonucleotide (cascade signal amplifier DNA 1 or cSA DNA 1) comprising, in the 5′ to 3′ direction, a second unique signal DNA generation sequence (E), an endonuclease recognition site (F), a sequence region comprising a sequence (G) that is complementary to at least a portion of the signal DNA generation sequence (E), and a sequence (H) that is homologous to the first signal DNA generation sequence (A) of the first SC DNA oligonucleotide, wherein sequence (H) optionally has at least one abasic site incorporated therein, and wherein at least a portion of the second signal DNA generation sequence (E) and the sequence (G) that is complementary to the signal DNA generation sequence (E) hybridize to form a hairpin structure; and a third oligonucleotide (cascade signal amplifier DNA 2 or cSA DNA 2) comprising, in the 5′ to 3′ direction, a third unique signal DNA generation sequence (I), an endonuclease recognition site (J), a sequence region comprising a sequence (K) that is complementary to at least a portion of the signal DNA generation sequence (I), and a sequence (L) that is homologous to the second signal DNA generation sequence (E) of the second oligonucleotide cSA DNA 1, wherein sequence (L) optionally has at least one abasic site incorporated therein, and wherein at least a portion of the third signal DNA generation sequence (I) and the sequence (K) that is complementary to the signal DNA generation sequence (I) hybridize to form a hairpin structure; a polymerase; and at least one endonuclease for a nicking reaction. In embodiments of this aspect, the method also comprises determining the presence or absence of a signal DNA, wherein the presence of the signal DNA indicates the presence of the target nucleic acid in the sample.

The method comprises contacting a sample with an endonuclease. The endonuclease may be a nicking endonuclease or a restriction endonuclease that is capable of or that can be used in nicking the sequence complementary to the endonuclease recognition site (B) within the SC DNA, the sequence complementary to the endonuclease recognition site (F) within the first cSA DNA 1, and the sequence complementary to the endonuclease recognition site (J) within the second cSA DNA 2. In some embodiments, the endonuclease comprises a nicking endonuclease or a restriction endonuclease that can catalyze or can be used to catalyze a double-stranded DNA nicking reaction. In embodiments providing a nicking endonuclease, the phosphodiester linkage of one strand of a double-strand DNA may be cleaved to generate a phosphate group on the 5′ side of the cleavage site and a hydroxyl group on the 3′ side. Non-limiting examples of nicking endonucleases include Nb.BbvCI, Nt.AlwI, Nt.BbvCI, Nb.BsrDI, Nb.Btsl, Nt.BspQI, Nt.BstNBI, Nb.BsmI, Nt.CviPII, and Nt.BsmAI.

In some embodiments, the endonuclease may be a restriction endonuclease. In these embodiments the restriction endonuclease recognition site may be modified so that the restriction endonuclease cleaves the phophodiester bond on only one strand of a double stranded DNA, and generates a nick in the double strand. Methods or strategies may be used to modify the activity of the restriction endonuclease such as, for example, including a chemical modification in at least one strand of a double-stranded nucleic acid that is not cleaved by the restriction enzyme. One non-limiting example of such a modification includes replacing the oxygen atom of phosphodiester linkage of one strand with a sulfur atom.

In embodiments providing a restriction endonuclease, the phosphodiester linkage of one strand of a double-strand DNA may be cleaved to generate a phosphate group on the 5′ side of the cleavage site and a hydroxyl group on the 3′ side. Non-limiting examples of restriction endonucleases include Hinc II, Hind II, Ava I, Fnu4HI, Tth111I and NciI.

The method comprises contacting a sample with a polymerase. In some embodiments, the polymerase may be a DNA polymerase having strand displacement activity. In some embodiments, the polymerase may be a polymerase that lacks 5′-3′ exonuclease activity, lacks 3′-5′ exonuclease activity, or lacks both 5′-3′ and 3′-5′ exonuclease activity. The polymerase may be eukaryotic, prokaryotic, or viral in origin, and can also be genetically modified. In some embodiments, the polymerase is selected from among those that function at lower temperatures, including ambient (e.g., room) temperatures. Non-limiting examples of DNA polymerases include Klenow fragments, DNA polymerase I derived from E. coli, 5′ to 3′ exonuclease-deficient Bst DNA polymerases derived from Bacillus stearothermophilus, and 5′ to 3′ exonuclease-deficient Bca DNA polymerases derived from Bacillus caldotenax.

One non-limiting embodiment of the methods disclosed herein is illustrated in FIGS. 2A, 2B, and 2C. Briefly, as illustrated in FIG. 2A, a sample is contacted with SC DNA in the presence of a DNA polymerase and an endonuclease capable of nicking the double-stranded form (i.e., complementary sequence) of the endonuclease recognition site (B), or a site adjacent to the double-stranded form (i.e., complementary sequence) of the endonuclease recognition site (B). If a target nucleic acid (T) is present in the sample, the 3′ end sequence of the target nucleic acid (T) hybridizes to the sequence (D) of the SC DNA (which is complementary to the target and which has at least one abasic site) and primes or initiates replication (by the DNA polymerase present in the reaction mixture) thereby generating double stranded extension sequence (X) that includes the double stranded endonuclease recognition site (B). Recognition of the newly-generated double stranded endonuclease recognition site (B) (by the endonuclease present in the reaction mixture), and subsequent nicking of the newly-generated strand (by the endonuclease present in the reaction mixture), generates a first oligonucleotide signal DNA 1 (S1) and extension sequence (X′). Because the 3′-OH of sequence (X′) at the nick serves as an initiation site for subsequent rounds of strand displacement replication, oligonucleotide (S1) is displaced from the SC DNA by DNA polymerase which continues to replicate and amplify signal DNA 1 (S1) in the reaction mixture.

As further illustrated in FIG. 2B, the first signal DNA (S1) produced by interaction of a Target (T) nucleic acid with a SC DNA can be converted to a second signal DNA 2 (S2) by the presence of a first cascade signal amplifier DNA 1 (or cSA DNA 1). Briefly, a first signal DNA 1 (S1) present in a reaction hybridizes to the sequence (H) of the first cSA DNA 1 (which is complementary to the signal DNA S1 and which optionally has at least one abasic site) and primes or initiates replication (by the DNA polymerase present in the reaction mixture) thereby generating double stranded extension sequence (Y) that includes the double stranded endonuclease recognition site (F). Recognition of the newly-generated double stranded endonuclease recognition site (F) (by endonuclease present in the reaction mixture), and subsequent nicking of the newly-generated strand (by endonuclease present in the reaction mixture), generates a different oligonucleotide signal sequence (S2) and extension sequence (Y′). Because the 3′-OH of sequence (Y′) at the nick serves as an initiation site for subsequent rounds of strand displacement replication, oligonucleotide (S2) is displaced from the cSA DNA 1 by DNA polymerase which continues to replicate and amplify a second unique signal DNA 2 (S2) in the reaction mixture.

As further illustrated in FIG. 2C, the second signal DNA (S2) produced by interaction of the first signal DNA (S1) with a first cascade SA DNA (cSA DNA 1) can be converted to a third signal DNA 3 (S3) by the presence of a second cascade signal amplifier DNA 2 (or cSA DNA 2). Briefly, a second signal DNA 2 (S2) present in a reaction hybridizes to the sequence (L) of the second cSA DNA 2 (which is complementary to the signal DNA S2 and which optionally has at least one abasic site) and primes or initiates replication (by the DNA polymerase present in the reaction mixture) thereby generating double stranded extension sequence (U) that includes the double stranded endonuclease recognition site (J). Recognition of the newly-generated double stranded endonuclease recognition site (J) (by endonuclease present in the reaction mixture), and subsequent nicking of the newly-generated strand (by endonuclease present in the reaction mixture), generates a different oligonucleotide signal sequence (S3) and extension sequence (U′). Because the 3′-OH of sequence (U′) at the nick serves as an initiation site for subsequent rounds of strand displacement replication, oligonucleotide (S3) is displaced from the cSA DNA 2 by DNA polymerase which continues to replicate and amplify a third unique signal DNA 3 (S3) in the reaction mixture.

In some embodiments, the Signal DNA (S2)/Target DNA ratio is from about 100 to about 1000, from about 100 to about 800, from about 100 to about 600, from about 100 to about 400, or from about 100 to about 200. In other embodiments, the Signal DNA (S3)/Target DNA ratio is from about 1000 to about 10000, from about 1000 to about 8000, from about 1000 to about 6000, from about 1000 to about 4000, or from about 1000 to about 2000.

Methods according to the invention may be performed under isothermal or substantially constant temperature conditions. In embodiments that relate to performing the method under a substantially constant temperature, some fluctuation in temperature is permitted. For example, in some embodiments a substantially constant temperature may fluctuate within a desired or identified target temperature range (e.g., about +/−2° C. or about +/−5° C.). In some embodiments, a substantially constant temperature may include temperatures that do not include thermal cycling. In some embodiments, methods can be performed at isothermal or substantially constant temperatures such as, for example, (1) temperatures at or below about the calculated/predicted or experimentally determined optimal hybridization or annealing temperature of the target nucleic acid (T) to sequence (D) of the SC DNA; (2) temperatures at or below the melting temperature of the target nucleic acid (T) bound to SC DNA (typically, hybridization or annealing temperatures are slightly below the melting temperature); (3) temperatures at or below the melting temperature of a signal DNA (S) bound to a cSA DNA; or (4) temperatures at or about the calculated/predicted or experimentally determined optimal reaction temperature for the polymerase and/or endonuclease present in the reaction mixture.

The methods may comprise reaction temperatures that range from about 20° C. to about 70° C., including lower temperatures falling within the range of about 20° C. to about 42° C. In some embodiments, the reaction temperature range is from 35° C. to 40° C. (e.g., 35° C., 36° C., 37° C., 38° C., 39° C., or 40° C.). In other embodiments, the reaction temperature is below 65° C., including lower temperatures below about 55° C., about 50° C., about 45° C., about 40° C., or about 30° C. In still other embodiments, reaction temperatures may be about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

The methods may be performed for a time that is adequate to allow for amplification of a detectable amount of signal sequence in the presence of a target nucleic acid. In some embodiments, the reaction time may range from about 5 minutes to 16 hours, or from about 3 minutes to 16 hours. In still other embodiments, the reaction time may range from about 5 to 120 minutes, or from about 15 to 60 minutes.

Because the various signal DNAs (S1), (S2), and (S3) are generated only in the presence of the target nucleic acid (T), methods according to the present invention detect the presence or absence of a target nucleic acid (T) in a sample by detecting the presence or absence of any one signal DNA. The signal DNAs (S1), (S2), and (S3) are different, and are not limited by sequence, and can be any sequence that is amenable to detection. The signal DNAs (S1), (S2), and (S3) are also not limited by length. Preferably, the signal DNAs (S1), (S2), and (S3) can be from about 5 to about 100 bases, and any integer between 5 and 100. In some embodiments, the signal DNAs (S1), (S2), and (S3) can be from about 5 to about 30 nucleic acid bases, and all integers between 5 and 30. In some embodiments, the signal DNAs (S1), (S2), and (S3) can be from about 10 to about 30 bases in length and all integers between 10 and 30. In yet further embodiments, the signal DNAs (S1), (S2), and (S3) can be from about 15 to about 30 bases in length and all integers between 15 and 30.

Methods according to the disclosure may be performed under buffer conditions that comprise a pH range from about 4 to about 10, or from about 7 to about 9. The buffer may comprise a salt concentration from about 10 mM to about 500 mM, or from about 50 mM to 150 mM. In some embodiments the method may be performed using an amount of SC and/or cSA DNAs that allows for amplification of a detectable amount of signal sequence in the presence of a target nucleic acid. In some embodiments, the SC and/or cSA DNA concentration may range from about 100 pM to about 100 pM, from about 1 nM to about 150 nM, from about 5 nM to about 50 nM, or from about 5 nM to about 25 nM.

In further embodiments, any method, oligonucleotide, composition, and/or kits disclosed herein may be used in combination with a chaotropic agent for the prevention or decrease of non-specific amplification of nucleic acids from a biological sample being tested for the presence or absence of a target nucleic acid in accordance with the methods disclosed herein. Chaotropic agents that can be used in combination with any one or more of the oligonucleotides, compositions, and kits disclosed herein include, without limitation, urea, formamide, guanidine hydrochloride, guanidine thiocyanate, sodium perchlorate, sodium iodide, or combinations thereof.

The presence of any one signal DNA (S1), (S2), and/or (S3) can be detected by any method known in the art. For example, gel electrophoresis and staining with ethidium bromide can be used. Also, the presence of any one signal DNA (S1), (S2), and/or (S3) can be detected using fluorescence polarization, immunoassay, fluorescence resonance energy transfer, enzyme labeling (such as peroxidase or alkaline phosphatase), fluorescent labeling (such as fluorescein or rhodamine), chemiluminescence, bioluminescence, surface plasmon resonance (SPR), or a fluorophore-modified probe DNA (e.g., TaqMan probe). The amplification product can also be detected by using a labeled nucleotide labeled with a biotin, for example. In such a case, the biotin in the amplification product can be detected using fluorescence-labeled avidin or enzyme-labeled avidin, for example. The amplification product can also be detected with electrodes by using redox intercalator known to those skilled in the art. The amplification product can also be detected using surface plasmon resonance (SPR), a Quarts Crystal Microbalance (QCM), or electrochemical methods (including those methods employing nanopore sensors).

The methods according to the present invention detect the presence or absence of a target nucleic acid (T) in a sample. The methods according to the present invention can also be used to quantitatively measure the concentration of a target nucleic acid in a test sample. For example, methods according to the present disclosure can be performed in the presence of a range of different known concentrations of the target nucleic acid, and calibration curves can be prepared and used as generally practiced in the art.

The target nucleic acid (T) in FIG. 2A can comprise any nucleic acid sequence and can include DNA, RNA, chemically modified nucleic acids, non-natural nucleic acids, nucleic acid analogs, or any hybrid or combination thereof. Accordingly, in some embodiments, DNA may include cDNA, genomic DNA, and synthetic DNA, and RNA may include total RNA, mRNA, rRNA, siRNA, hnRNA, piRNA, aRNA, miRNA, and synthetic RNA. While some embodiments relate to particular target nucleic acid sequences, any nucleic acid sequence, including auxiliary nucleic acid sequence, can be a target nucleic acid sequence to be detected. The disclosure provides for the detection of a target nucleic acid with selectivity and sensitivity even when the nucleic acid is a short-chain nucleic acid. Accordingly, the degree of complementarity between sequence (D) of the SC DNA and target nucleic acid (T) allows for specific hybridization between the sequences (e.g., the number of complementary nucleotides in sequence (D) of the sequence conversion DNA and target nucleic acid (T) sequences avoids non-specific hybridization under a given set of reaction conditions).

In embodiments, the target nucleic acid sequence can be from, or derived from any number of sources including, for example, genomic DNA, expressed mRNA, nucleic acid sequences from pathogens (microbes, viruses), or therapeutic nucleic acids. Accordingly, the SC and cSA DNAs and the methods disclosed herein may be used for the diagnosis and prognosis of diseases (e.g., arising from genetic and infectious sources), identification of contaminants (e.g., food-borne illnesses, equipment contamination), personalized medicine (e.g., monitoring and/or prognosis of a therapy), and the like. For example, molecular diagnostic testing can be performed with respect to the following infectious diseases: Hepatitis B Virus (HBV); hepatitis C (HCV); HCV (genotypes 1-6); Human Immunodeficiency Virus type 1 (HIV-1); Chlamydia trachomatis; Neisseria gonorrhoeae; influenza A; influenza B; Respiratory Syncytial Virus (RSV); and Parvo virus.

In some embodiments, the target nucleic acid can comprise microRNAs (miRNA). microRNAs include small non-coding RNA molecules of about 22 nucleotides. microRNAs are known to function in transcription and post-transcriptional regulation of gene expression. It is known that microRNAs function by base pairing with complementary regions of messenger RNA (mRNA), resulting in gene silencing via translational repression or target degradation.

Any type of sample that may comprise a target nucleic acid may be used in the methods disclosed herein. As such, the sample containing or suspected of containing a target nucleic acid is not specifically limited, and includes, for example, biological samples derived from living subjects, such as whole blood, serum, buffy coat, urine, feces, cerebrospinal fluid, seminal fluid, saliva, tissue (such as cancerous tissue or lymph nodes), cell cultures (such as mammalian cell cultures or bacterial cultures); samples containing nucleic acids, such as viroids, viruses, bacteria, fungi, yeast, plants, and animals; samples (such as food and biological preparations) that may contain or be infected with microorganisms such as viruses or bacteria; and samples that may contain biological substances, such as soil, industrial process and manufacturing equipment, and wastewater; and samples derived from various water sources (e.g., drinking water). Furthermore, a sample may be processed by any known method to prepare a nucleic acid-containing composition used in the methods disclosed herein. Examples of such preparations can include cell breakage (e.g., cell lysates and extracts), sample fractionation, nucleic acids in the samples, and specific nucleic acid molecular groups such as mRNA-enriched samples. The sample used in the method for detecting a target nucleic acid of the present invention is not limited to those derived from biological and natural products as mentioned above and may be a sample containing a synthetic oligonucleotide.

Methods according to the present invention can be performed in combination with the Abbott m2000sp sample preparation system. The m2000sp uses magnetic particle technology to capture nucleic acids and washes the particles to remove unbound sample components. The bound nucleic acids are eluted and transferred to a 96 deep-well plate. The Abbott m2000sp can also combine with the washed nucleic acids transferred to the 96 deep-well plate any reagents required to perform the methods according to the present technology. For example, SC and cSA DNAs, polymerases, endonucleases, molecular beacons, and any other reagent (e.g., dNTPs) can be added as required, or desired.

Methods according to the present invention can also be interfaced with point-of-care platforms. For example, the incorporation of a deoxyribonucleotide triphosphate (dNTP) into a growing DNA strand involves the formation of a covalent bond and the release of pyrophosphate and a positively charged hydrogen ion affecting the pH of a reaction. As such, the synthesis of signal DNA according to methods of the present invention can be detected by tracking changes in pH using, for example, point-of-care micro-pH meters. For example, Abbott's i-STAT point-of-care system can be supplied with single-use disposable cartridges containing micro fabricated sensors, calibration solutions, fluidic systems, and waste chambers for analysis of pH.

The methods disclosed herein can comprise additional reagents. Some non-limiting examples of other reagents that can be used in the nucleic acid amplification reaction include metallic salts such as sodium chloride, magnesium chloride, magnesium acetate, and magnesium sulfate; substrates such as dNTP mix; and buffer solutions such as Tris-HCl buffer, tricine buffer, sodium phosphate buffer, and potassium phosphate buffer. Likewise, detergents, oxidants and reducing agents can also be used in the practice of the methods disclosed herein. Furthermore, agents such as dimethyl sulfoxide and betaine (N, N, N-trimethylglycine); acidic substances described in International Publication No. WO 99/54455; and cationic complexes can be used.

The methods and nucleic acid structures provided herein may be used in combination with other methods to provide for the exponential amplification of a signal DNA in the presence of a target nucleic acid. For example, the methods and compositions according to the present disclosure may be used in combination with covered sequence conversion DNAs, as described in U.S. patent application Ser. No. 14/597,981, entitled “Covered Sequence Conversion DNA and Detection Methods” which is incorporated herein by reference. The methods and compositions according to the present disclosure may also be used in combination with chemically modified sequence conversion and signal amplifier DNAs, as described in U.S. patent application Ser. No. 14/882,124, entitled “Sequence Conversion and Signal Amplifier DNA Having Locked Nucleic Acids and Detection Methods Using Same” which is incorporated herein by reference. The methods and compositions according to the present disclosure may also be used in combination with a poly DNA spacer sequence conversion and signal amplifier DNAs, as described in U.S. patent application Ser. No. 14/882,109, entitled “Sequence conversion and signal amplifier DNA having poly DNA spacer sequences and detection methods using same” which is incorporated herein by reference. The methods and compositions according to the present invention may also be used in combination with the methods and compositions described in U.S. patent application Ser. No. 14/998,162, entitled “Sequence Conversion and Signal Amplifier DNA Cascade Reactions and Detection Methods Using Same” which is incorporated herein by reference.

The term “about” generally refers to a range of numbers that one of skill in the art would consider equivalent to the recited value (i.e., having the same function or result). The term “about”, as used herein, is intended to refer to ranges that include approximately 10-20% greater than or less than the referenced value. In certain circumstances, one of skill in the art will recognize that, due to the nature of the referenced value, the term “about” can mean more or less than a 10-20% deviation from that value.

The Examples that follow are intended to be illustrative of the aspects and embodiments described above. Neither the above disclosure nor the Examples below should be viewed as limiting to the scope of the appended claims. One of skill in the art will appreciate that the disclosure is not limited by the particular terminology which is used to describe and illustrate the various aspects of the disclosure.

### Example 1

A set of reactions was performed in accordance with the present disclosure using SC DNA 1 having the sequence 5′-TCTTGCGTAT CTTCTCCACC TCAGCGCAAG ATCAACATCA GTCTGATAAG CTA-idT-idT-3′ (SEQ ID NO.:1) or SC DNA 2 having the sequence 5′-TCTTGCGTAT CTTCTCCACC TCAGCGCAAG ATCAACAααA GTCTGATAAG CTA-idT-idT-3′ (SEQ ID NO.: 2) with dSpacer abasic artificial nucleic acids (“α”) at positions 38 and 39 from the 5′ end. The SC DNAs were present in the reaction at 1.4 nM. Ten different normal serum samples were tested for the presence of non-specific background signal amplification (i.e., ten serum samples lacking target nucleic acid).

Another set of reactions were performed in accordance with the present disclosure using the same SC DNA 1 and SC DNA 2 detailed above. The SC DNAs were present in the reaction at 1.4 nM. The reactions were performed in the presence of 0, 100, or 1000 fM model RNA target nucleic acid (i.e., RNA having the equivalent sequence of microRNA-21).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst Large Fragment (L.F.), present at 0.02 U/uL, and the nicking endonuclease Nb.BbvCl, present at 0.025 U/uL. The total amplification time was 25 min. The generated signal DNA was detected by chemiluminescent measurement using ARCHITECT.

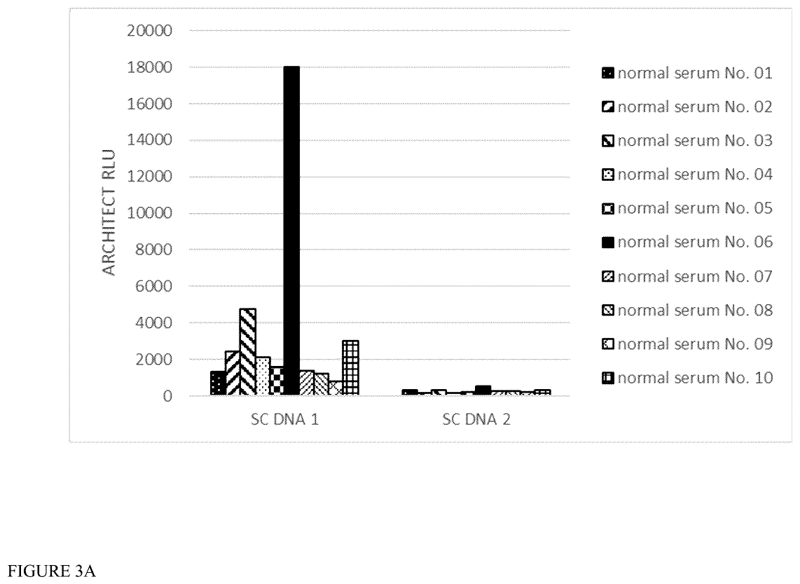

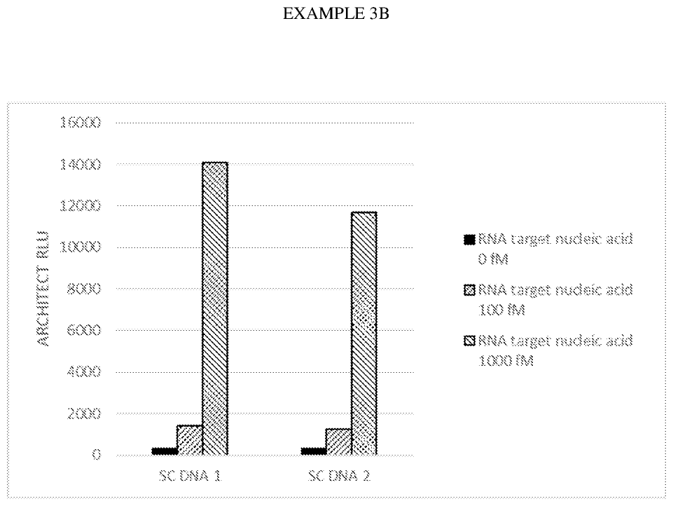

The results of each reaction set of Example 1 are shown in FIGS. 3A (serum) and 3B (added target miRNA), respectively.

### Example 2

Another set of reactions were performed in accordance with the present disclosure, using the following SC DNAs: SC DNA 1 having the sequence 5′-TCTTGCGTAT CTTCTCCACC TCAGCGCAAG ATCAACATCA GTCTGATAAG CTA-idT-idT-3′ (SEQ ID NO.: 1), SC DNA 2 having the sequence 5′-TCTTGCGTAT CTTCTCCACC TCAGCGCAAG ATCAACAααA GTCTGATAAG CTA-idT-idT-3′ (SEQ ID NO.: 2) with dSpacer abasic artificial nucleic acids (“α”) at positions 38 and 39 from the 5′ end, or SC DNA 3 having the sequence 5′-TCTTGCGTAT CTTCTCCACC TCAGCGCAAG ATCAACAPPA GTCTGATAAG CTA-idT-idT-3′ (SEQ ID NO.: 3) with ethynyl dSpacer abasic artificial nucleic acids (“β”) at positions 38 and 39 from the 5′ end. The SC DNAs were present in the reaction at 1.4 nM. Ten different normal serum samples (purchased from a commercial source) were tested for the presence of non-specific background signal amplification (i.e., ten serum samples lacking target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

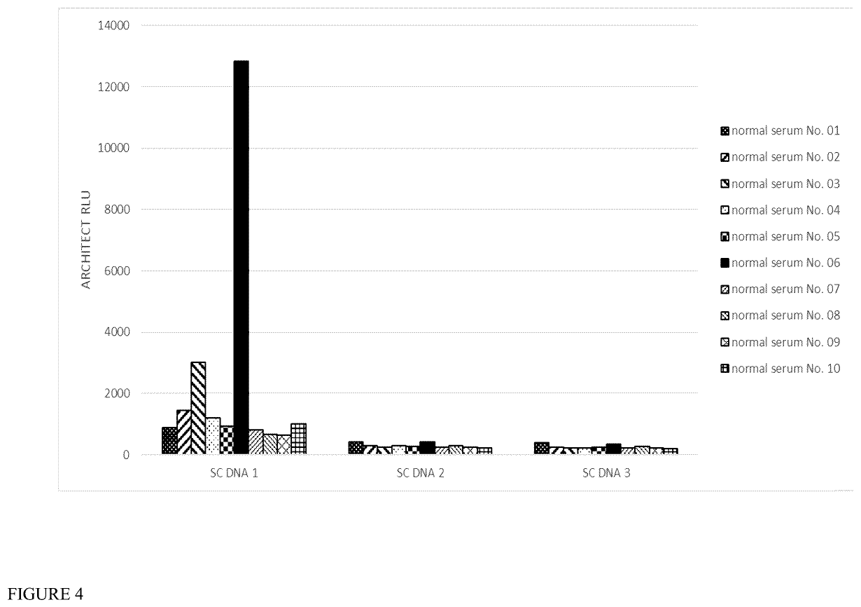

The results of Example 2 are illustrated in FIG. 4.

### Example 3

Another set of reactions were performed in accordance with the present disclosure using SC DNA 1 (SEQ ID NO.: 1), SC DNA 2 (SEQ ID NO.: 2), or SC DNA 3 (SEQ ID NO.: 3). The SC DNAs were present in the reaction at 1.4 nM. The reactions were performed in the presence of 0, 10, 100, or 1000 fM model DNA target nucleic acid (i.e., DNA having the equivalent sequence of microRNA-21).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

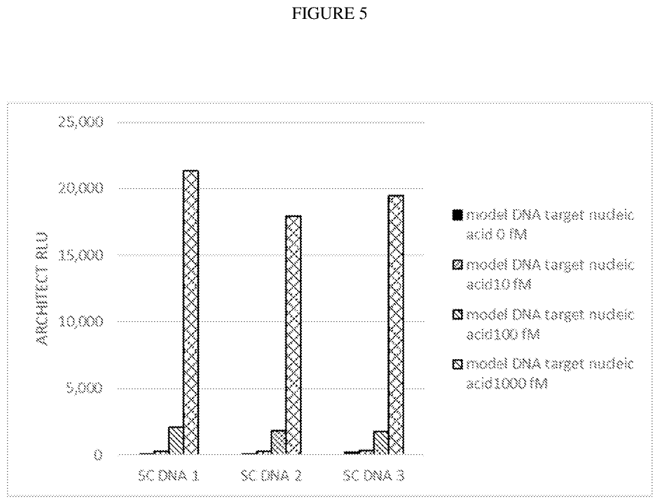

The results of Example 3 are illustrated in FIG. 5.

### Example 4

Another set of reactions were performed in accordance with the present disclosure using SC DNA 1 (SEQ ID NO.: 1), SC DNA 2 (SEQ ID NO.: 2), or SC DNA 3 (SEQ ID NO.: 3). The SC DNAs were present in the reaction at 1.4 nM. The reactions were performed in the presence of 0, 10, 100, or 1000 fM microRNA-21 target nucleic acid (i.e., RNA target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

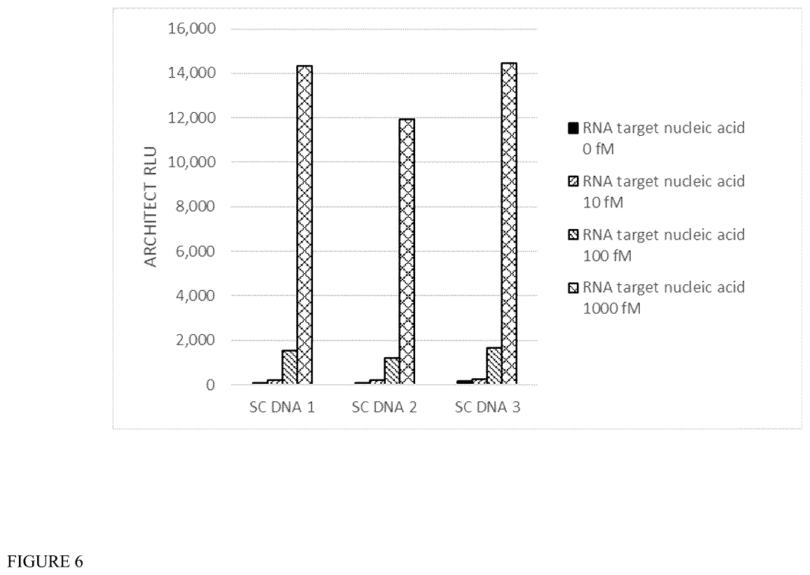

The results of Example 4 are illustrated in FIG. 6.

### Example 5

Another set of reactions were performed in accordance with the present disclosure using SC DNA 1 (SEQ ID NO.: 1); SC DNA 2 (SEQ ID NO.: 2); SC DNA 4 having the sequence 5′-TCTTGCGTAT CTTCTCCACC TCAGCGCAAG ATCAACAαCA GTCTGATAAG CTA-idT-idT-3′ (SEQ ID NO.: 4) with a dSpacer abasic artificial nucleic acid (“α”) at position 38 from the 5′ end; SC DNA 5 having the sequence 5′-TCTTGCGTAT CTTCTCCACC TCAGCGCAAG ATCAACAααα GTCTGATAAG CTA-idT-idT-3′ (SEQ ID NO.: 5) with dSpacer abasic artificial nucleic acids (“α”) at positions 38, 39, and 40 from the 5′ end; or SC DNA 6 having the sequence 5′-TCTTGCGTAT CTTCTCCACC TCAGCGCAAG ATCAACAααα αTCTGATAAG CTA-idT-idT-3′ (SEQ ID NO.: 6) with dSpacer abasic artificial nucleic acids (“α”) at positions 38, 39, 40, and 41 from the 5′ end. The SC DNAs were present in the reaction at 1.4 nM. Ten different normal serum samples were tested for the presence of non-specific background signal amplification (i.e., ten serum samples lacking target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

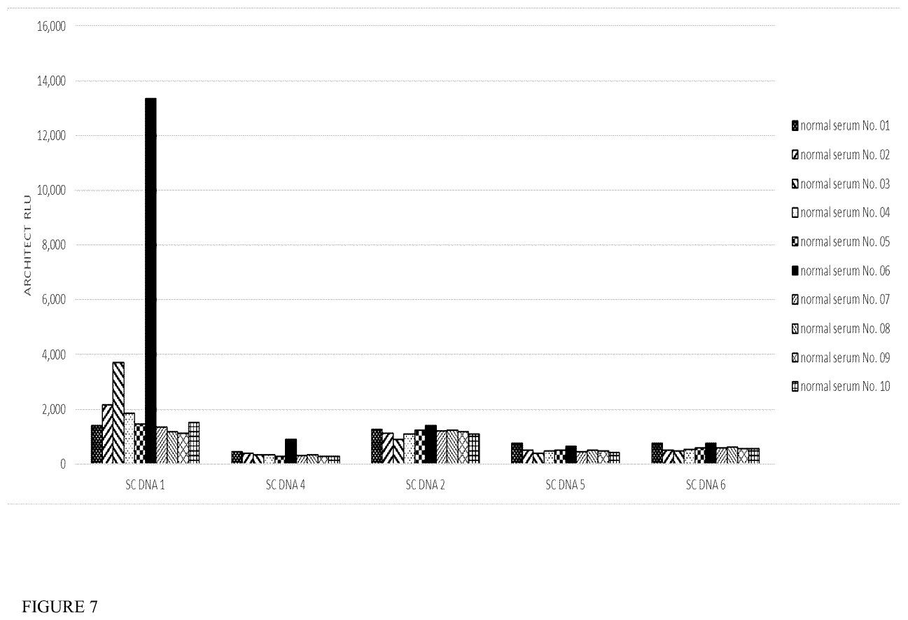

The results of Example 5 are illustrated in FIG. 7.

### Example 6

Another set of reactions were performed in accordance with the present disclosure using SC DNA 1 (SEQ ID NO.: 1), SC DNA 2 (SEQ ID NO.: 2), SC DNA 4 (SEQ ID NO.: 4); SC DNA 5 (SEQ ID NO.: 5); and SC DNA 6 (SEQ ID NO.: 6). The SC DNAs were present in the reaction at 1.4 nM. The reactions were performed in the presence of 0, 10, 100, or 1000 fM model DNA target nucleic acid (i.e., DNA having the equivalent sequence of microRNA-21).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

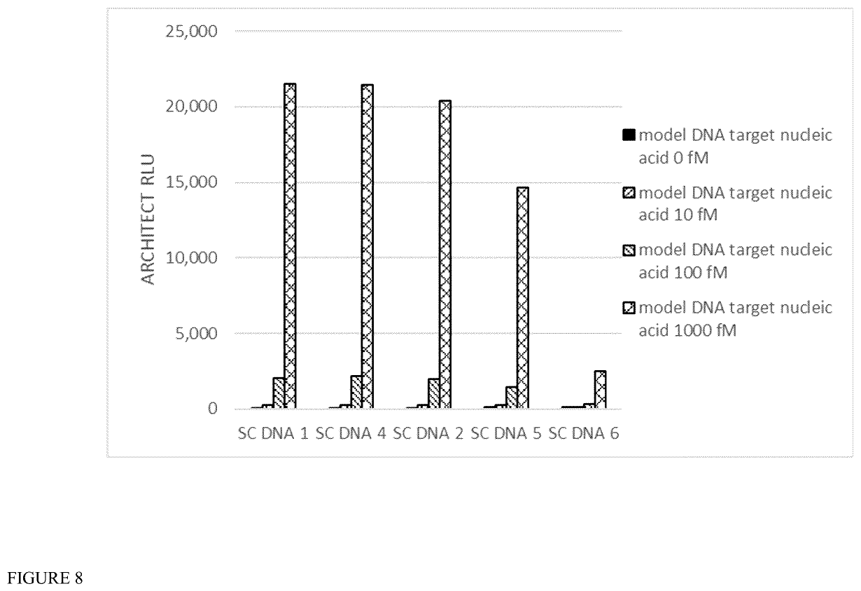

The results of Example 6 are illustrated in FIG. 8.

### Example 7

Another set of reactions were performed in accordance with the present disclosure using SC DNA 1 (SEQ ID NO.: 1), SC DNA 2 (SEQ ID NO.: 2), SC DNA 4 (SEQ ID NO.: 4); SC DNA 5 (SEQ ID NO.: 5); and SC DNA 6 (SEQ ID NO.: 6). The SC DNAs were present in the reaction at 1.4 nM. The reactions were performed in the presence of 0, 10, 100, or 1000 fM microRNA-21 target nucleic acid (i.e., RNA target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

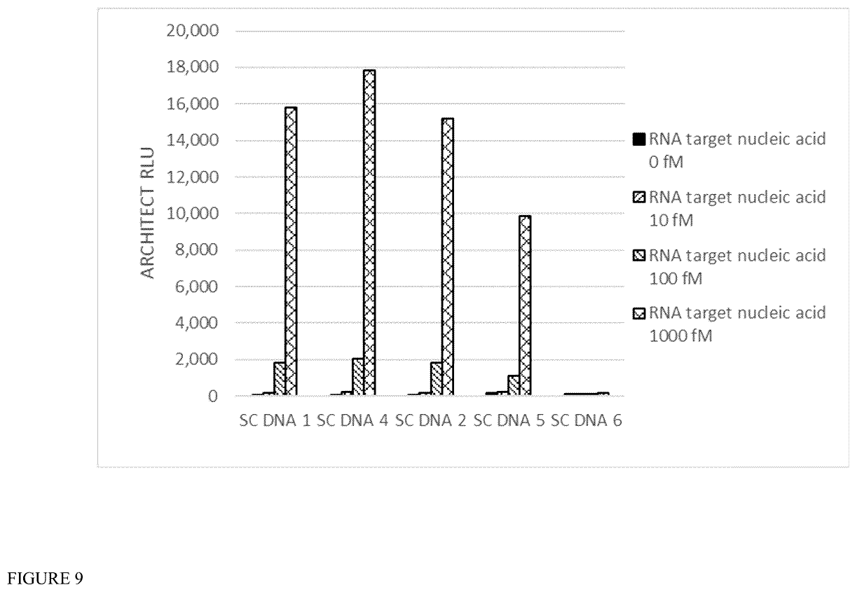

The results of Example 7 are illustrated in FIG. 9.

### Example 8

Another set of reactions were performed in accordance with the present disclosure using SC DNA 1 (SEQ ID NO.: 1), SC DNA 2 (SEQ ID NO.: 2), SC DNA 7 having the sequence 5′-TCTTGCGTAT CTTCTCCACC TCAGCGCAAG ATCAACATCα αTCTGATAAG CTA-idT-idT-3′ (SEQ ID NO.: 7) with dSpacer abasic artificial nucleic acids (“α”) at positions 40 and 41 from the 5′ end, SC DNA 8 having the sequence 5′-TCTTGCGTAT CTTCTCCACC TCAGCGCAAG ATCAACATαα GTCTGATAAG CTA-idT-idT-3′ (SEQ ID NO.: 8) with dSpacer abasic artificial nucleic acids (“α”) at positions 39 and 40 from the 5′ end, SC DNA 9 having the sequence 5′-TCTTGCGTAT CTTCTCCACC TCAGCGCAAG ATCAACααCA GTCTGATAAG CTA-idT-idT-3′ (SEQ ID NO.: 9) with a dSpacer abasic artificial nucleic acids (“α”) at positions 37 and 38 from the 5′ end, SC DNA 10 having the sequence 5′-TCTTGCGTAT CTTCTCCACC TCAGCGCAAG ATCAAααTCA GTCTGATAAG CTA-idT-idT-3′ (SEQ ID NO.: 10) with a dSpacer abasic artificial nucleic acids (“α”) at positions 36 and 37 from the 5′ end, and SC DNA 11 having the sequence 5′-TCTTGCGTAT CTTCTCCACC TCAGCGCAAG ATCAααATCA GTCTGATAAG CTA-idT-idT-3′ (SEQ ID NO.: 11) with a dSpacer abasic artificial nucleic acids (“α”) at positions 35 and 36 from the 5′ end. The SC DNAs were present in the reaction at 1.4 nM. Ten different normal serum samples were tested for the presence of non-specific background signal amplification (i.e., ten serum samples lacking target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

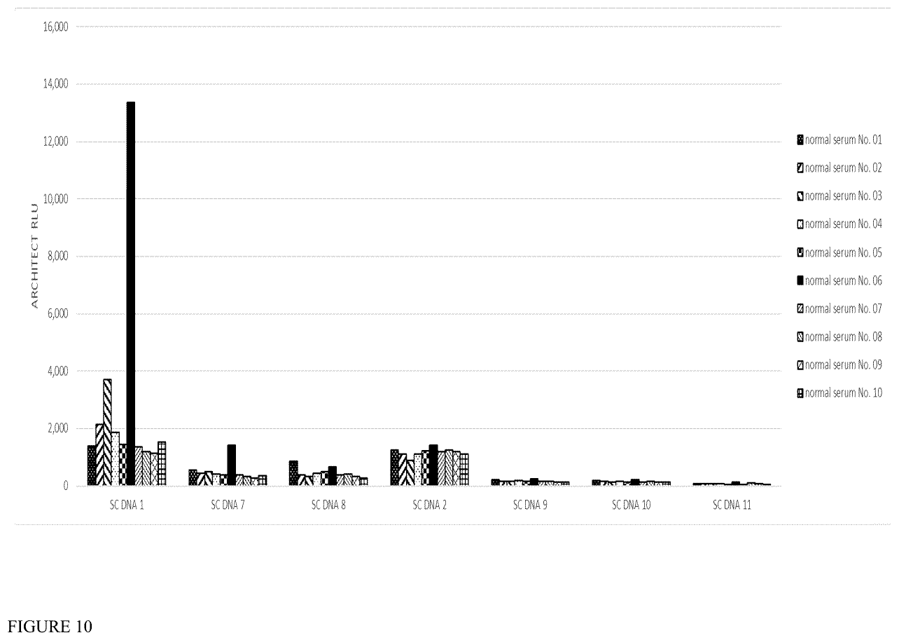

The results of Example 8 are presented in FIG. 10.

### Example 9

Another set of reactions were performed in accordance with the present disclosure using SC DNA 1 (SEQ ID NO.: 1), SC DNA 2 (SEQ ID NO.: 2), SC DNA 7 (SEQ ID NO.: 7), SC DNA 8 (SEQ ID NO.: 8), SC DNA 9 (SEQ ID NO.: 9), SC DNA 10 (SEQ ID NO.: 10), and SC DNA 11 (SEQ ID NO.: 11). The SC DNAs were present in the reaction at 1.4 nM. The reactions were performed in the presence of 0, 10, 100, or 1000 fM model DNA target nucleic acid (i.e., DNA having the equivalent sequence of microRNA-21).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

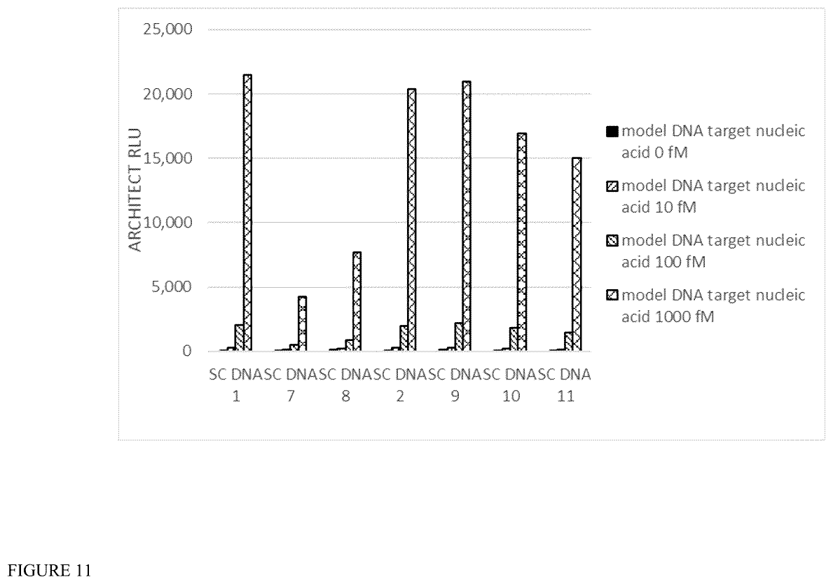

The results of Example 9 are illustrated in FIG. 11.

### Example 10

Another set of reactions were performed in accordance with the present disclosure using SC DNA 1 (SEQ ID NO.: 1), SC DNA 2 (SEQ ID NO.: 2), SC DNA 7 (SEQ ID NO.: 7), SC DNA 8 (SEQ ID NO.: 8), SC DNA 9 (SEQ ID NO.: 9), SC DNA 10 (SEQ ID NO.: 10), and SC DNA 11 (SEQ ID NO.: 11). The SC DNAs were present in the reaction at 1.4 nM. The reactions were performed in the presence of 0, 10, 100, or 1000 fM microRNA-21 target nucleic acid (i.e., RNA target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

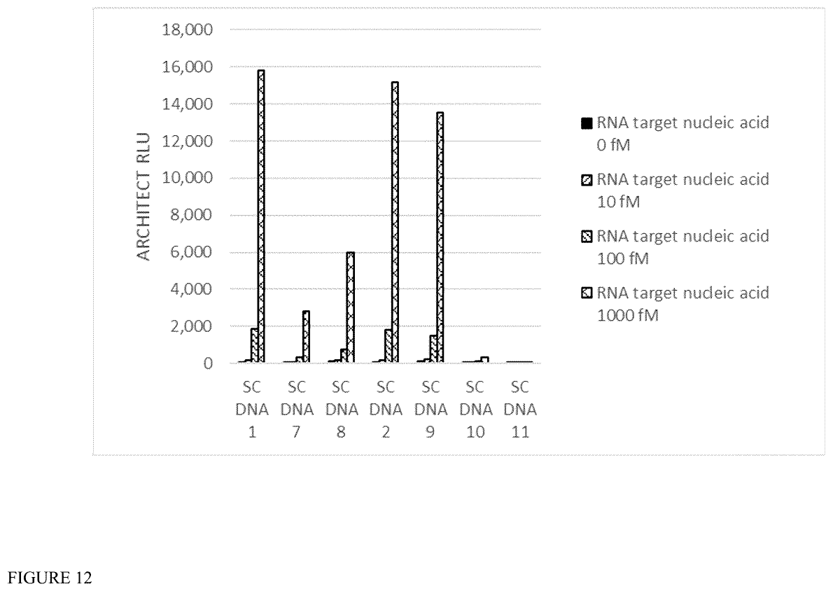

The results of Example 10 are illustrated in FIG. 12.

### Example 11

Another set of reactions were performed in accordance with the present disclosure using SC DNA 1 (SEQ ID NO.: 1), SC DNA 2 (SEQ ID NO.: 2), and SA DNA 1 having the sequence 5′-AGCAGCAACA TAGCAGAACC TCAGCGCTGC TTCTTGCααA TCTTCTCCA-idT-idT-3′ (SEQ ID NO.: 12) with dSpacer abasic artificial nucleic acids (“α”) at positions 38 and 39 from the 5′ end. The SC DNAs were present in the reaction at 0.14 nM, and the SA DNA 1 were present in the reaction at 1.4 nM. Ten different normal serum samples were tested for the presence of non-specific background signal amplification (i.e., ten serum samples lacking target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

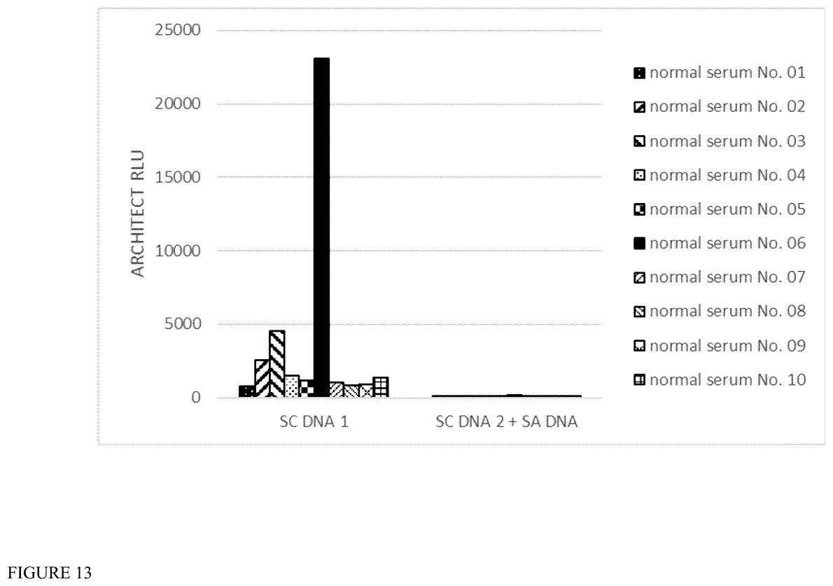

The results of Example 11 are illustrated in FIG. 13.

### Example 12

Another set of reactions were performed in accordance with the present disclosure using SC DNA 1 (SEQ ID NO.: 1), SC DNA 2 (SEQ ID NO.: 2), and SA DNA 1 (SEQ ID NO.: 12). The SC DNAs were present in the reaction at 0.14 nM, and the SA DNA 1 were present in the reaction at 1.4 nM. The reactions were performed in the presence of 0, 100, or 1000 fM model DNA target nucleic acid (i.e., DNA having the equivalent sequence of microRNA-21).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

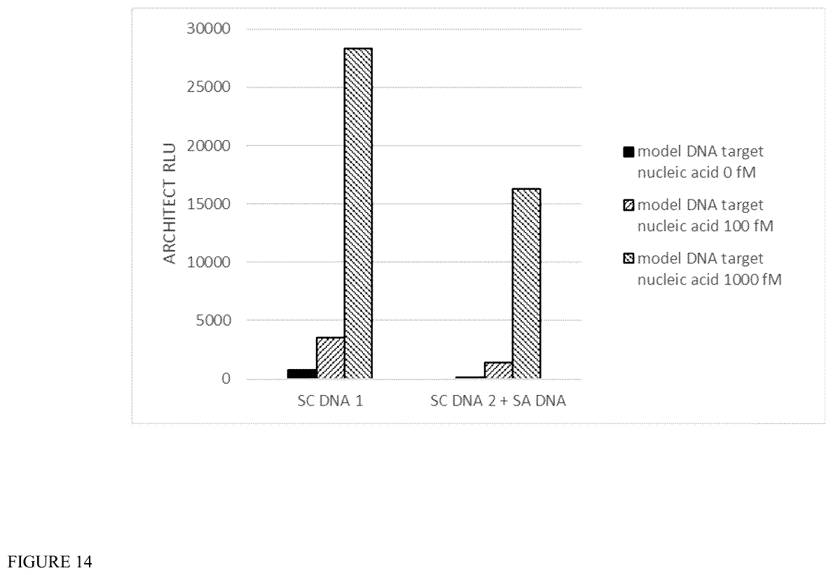

The results of Example 12 are illustrated in FIG. 14.

### Example 13

Another set of reactions were performed in accordance with the present disclosure using SC DNA 1 (SEQ ID NO.: 1), SC DNA 2 (SEQ ID NO.: 2), and SA DNA 1 (SEQ ID NO.: 12). The SC DNAs were present in the reaction at 0.14 nM, and the SA DNA 1 were present in the reaction at 1.4 nM. The reactions were performed in the presence of 0,100, or 1000 fM microRNA-21 target nucleic acid (i.e., RNA target nucleic acid).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

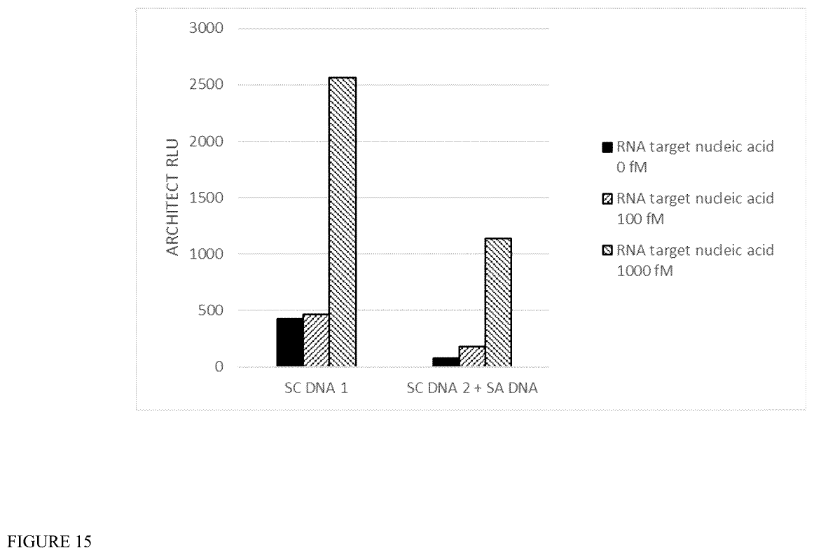

The results of Example 13 are illustrated in FIG. 15.

### Example 14

A series of reactions were performed in accordance with the present disclosure to compare the effects of SC DNA and/or SA DNA having abasic sites incorporated therein on non-specific background signal amplification. Reactions were performed on 58 normal serum samples (i.e. lacking target nucleic acid). Each serum sample was analyzed using the following two reaction conditions:

Reaction Condition A used SC DNA 13 having the sequence 5′-AGCAGCAACA TAGCAGAACC TCAGCTTCTG CTATGTTGCT GCTTCAACAT AAGTCTGATA AGCTA-idT-idT-3′ (SEQ ID NO.: 15) and SA DNA 3 having the sequence 5′-TCTTGCGTAT CTTCTCCACC TCAGCATACG CAAGAAGCAG CAACATAGCA GAA-idT-idT-3′ (SEQ ID NO.: 16). SC and SA DNA were present in the reaction at 0.42 nM and 1.4 nM, respectively.

Reaction Condition B used SC DNA 12 having the sequence 5′-AGCAGCAACA TAGCAGAACC TCAGCTTCTG CTATGTTGCT GCTTCAACAα αAGTCTGATA AGCTA-idT-idT-3′-idT-idT-3′ (SEQ ID NO.: 13) with dSpacer abasic artificial nucleic acids (“α”) at positions 50 and 51 and SA DNA 2 having the sequence 5′-TCTTGCGTAT CTTCTCCACC TCAGCATACG CAAGAAGCAG ααACATAGCA GAA-idT-idT-3′ (SEQ ID NO.: 14) with dSpacer abasic artificial nucleic acids (“α”) at positions 41 and 42. SC and SA DNA were present in the reaction at 0.42 nM and 1.4 nM, respectively.

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst L.F., present at 0.02 U/uL, and the nicking endonuclease Nb.BbvCl, present at 0.025 U/uL. The total amplification time was 25 min. The generated signal DNA was detected by chemiluminescent measurement using ARCHITECT.

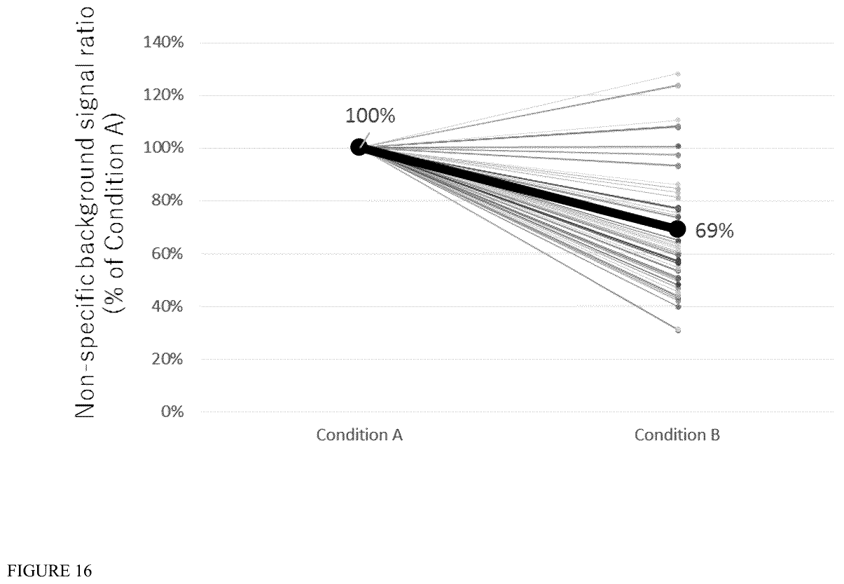

The results of Example 14 are shown in FIGS. 16-18 (gray lines represent individual biological replicates and black lines represent the average of all biological replicates). FIG. 16 illustrates the effect of SC DNA and SA DNA, each having abasic sites incorporated therein, on non-specific background signal. These data demonstrate SC DNA and SA DNA, each comprising abasic sites, (Reaction Condition B) can reduce non-specific background signal as compared to reactions in which SC DNA and SA DNA lack abasic sites (Reaction Condition A).

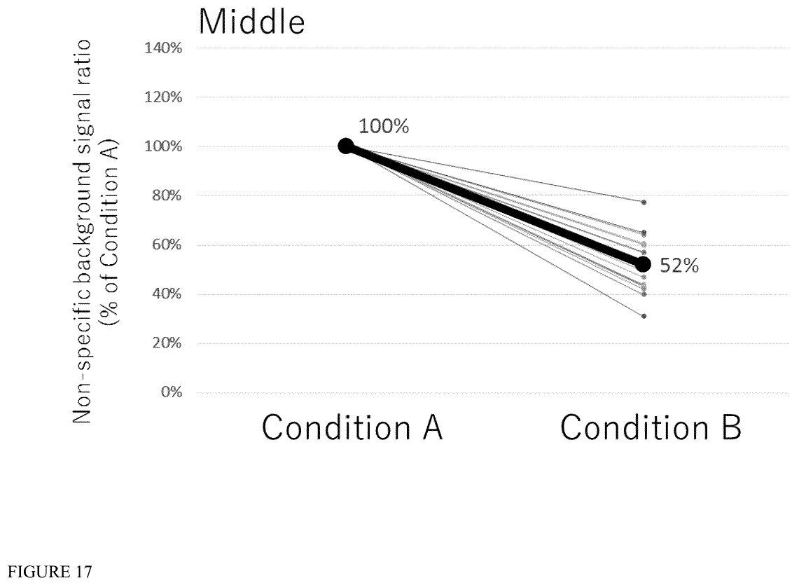

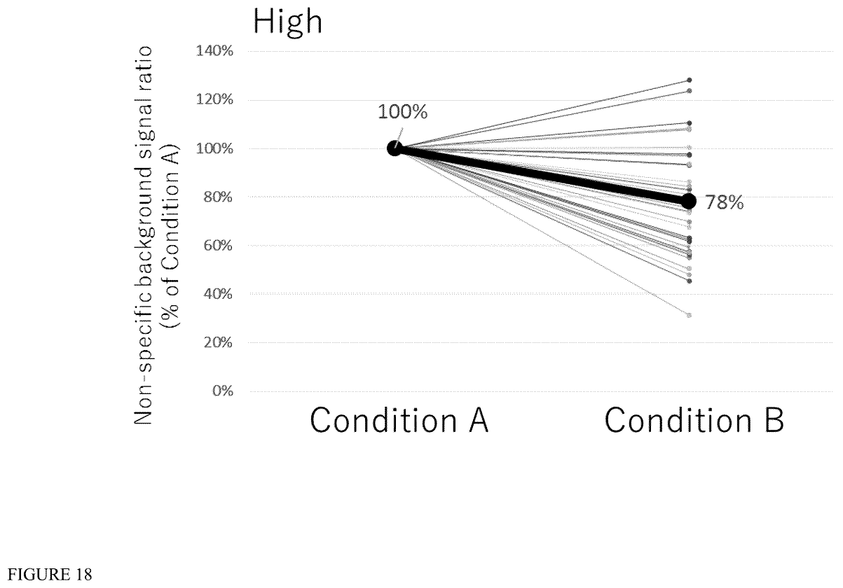

FIG. 17 illustrates the effect of SC DNA and SA DNA, each having abasic sites incorporated therein, on non-specific background signal from 20 normal serums reporting mid-range false positive values (measured ARCHITECT values were less than 2000 relative light units). FIG. 18 illustrates the effect of SC DNA and SA DNA, each having abasic sites incorporated therein, on non-specific background signal from 38 normal serums reporting high-range false positive values (measured ARCHITECT values were greater than 2000 relative light units). These data demonstrate SC DNA and SA DNA, each having abasic sites incorporated therein, have a greater effect on non-specific background signal reduction in normal serums reporting mid-range false positive values as compared to those reporting high-range false positive values.

### Example 15

A set of reactions was performed in accordance with the present disclosure to examine the effect of varying the length of the sequence that is complementary to at least a portion of the signal DNA generation sequence of a SC DNA on specific and non-specific amplification. The reactions were performed using SC DNA 1 having the sequence 5′-TCTTGCGTAT CTTCTCCACC TCAGCGCAAG ATCAACATCA GTCTGATAAG CTA-idT-idT-3′ (SEQ ID NO.:1), SC DNA 2 having the sequence 5′-TCTTGCGTAT CTTCTCCACC TCAGCGCAAG ATCAACAααA GTCTGATAAG CTA-idT-idT-3′ (SEQ ID NO.: 2) with dSpacer abasic artificial nucleic acids (“α”) at positions 38 and 39 from the 5′ end, SC DNA 14 having the sequence 5′-TCTTGCGTAT CTTCTCCACC TCAGCATACG CAAGATCAACAα αAGTCTGATA AGCTA-idT-idT-3′ (SEQ ID NO.: 17) with dSpacer abasic artificial nucleic acids (“α”) at positions 42 and 43 from the 5′ end, SC DNA 15 having the sequence 5′-TCTTGCGTAT CTTCTCCACC TCAGCGAAGATACGCAAG ATCAACAα αAGTCTGATA AGCTA-idT-idT-3′ (SEQ ID NO.: 18) with dSpacer abasic artificial nucleic acids (“α”) at positions 46 and 47 from the 5′ end, and SC DNA 16 having the sequence 5′-TCTTGCGTAT CTTCTCCACC TCAGCTGGAGAAGATACGCAAG ATCAACAα αAGTCTGATA AGCTA-idT-idT-3′ (SEQ ID NO.: 19) with dSpacer abasic artificial nucleic acids (“α”) at positions 50 and 51 from the 5′ end. The SC DNAs were present in the reaction at 1.4 nM. Ten different normal serum samples were tested for the presence of non-specific background signal amplification (i.e., ten serum samples lacking target nucleic acid).

Another set of reactions were performed in accordance with the present disclosure using SC DNA 1 (SEQ ID NO.: 1), SC DNA 2 (SEQ ID NO.: 2), or SC DNA 14 (SEQ ID NO.: 17), SC DNA 15 (SEQ ID NO.: 18), or SC DNA 16 (SEQ ID NO.: 19), detailed above. The SC DNAs were present in the reaction at 1.4 nM. The reactions were performed in the presence of 0, 10, 100, or 1000 fM model DNA target nucleic acid having the sequence 5′-tagcttatca gactgatgtt ga-3′ (SEQ ID NO.: 20) (i.e., DNA having the equivalent sequence of microRNA-21).

Another set of reactions were performed in accordance with the present disclosure using SC DNA 1 (SEQ ID NO.: 1), SC DNA 2 (SEQ ID NO.: 2), or SC DNA 14 (SEQ ID NO.: 17), SC DNA 15 (SEQ ID NO.: 18), or SC DNA 16 (SEQ ID NO.: 19), detailed above. The SC DNAs were present in the reaction at 1.4 nM. The reactions were performed in the presence of 0, 10, 100, or 1000 fM model RNA target nucleic acid having the sequence 5′-uagcuuauca gacugauguu ga-3′ (SEQ ID NO.: 21) (i.e., RNA having the equivalent sequence of microRNA-21).

Reactions were performed at 37° C. in a 120 uL reaction solution containing CutSmart Buffer having 50 mM K-Acetate, 20 mM Tris-Acetate, 7.5 mM Mg-Acetate, 0.1 mg/mL BSA, 1 mM DTT (pH 7.9), the DNA polymerase Bst Large Fragment (L.F.), present at 0.02 U/uL, and the nicking endonuclease Nb.BbvCl, present at 0.025 U/uL. The total amplification time was 25 min. The generated signal DNA was detected by chemiluminescent measurement using ARCHITECT.

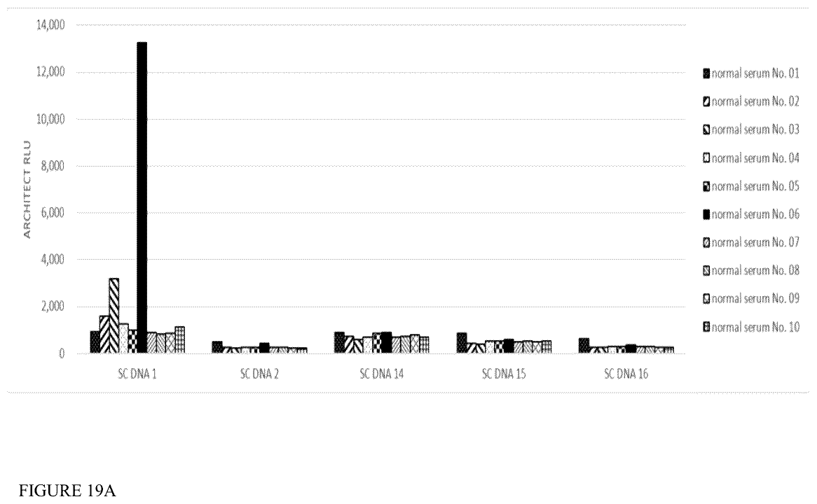

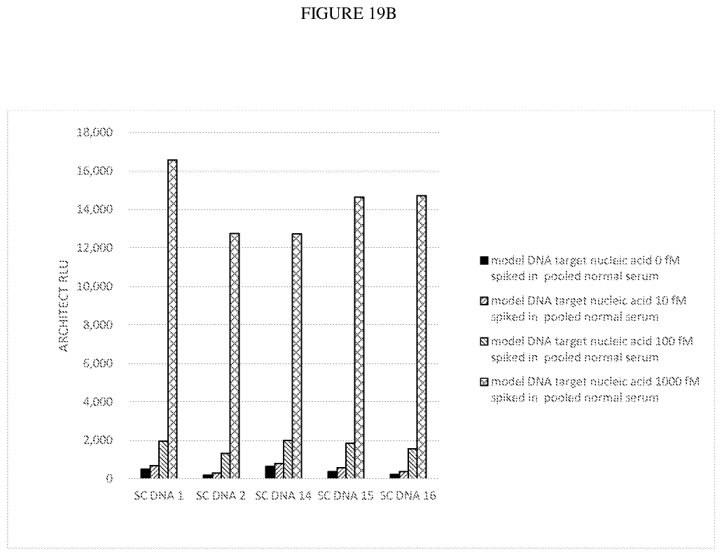

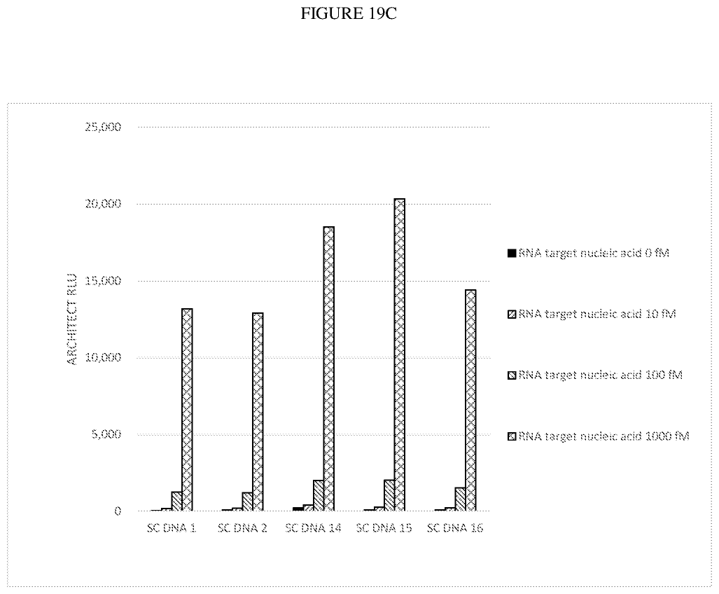

The results of each reaction set of Example 15 are shown in FIGS. 19A (serum), 19B (added target DNA), and 19C (added target RNA), respectively.

Tables 1 and 2 below disclose the SEQ ID NO. of the sequences disclosed herein.

TABLE 1

| SEQ | | |
|---|---|---|
| ID | | |
| NO. | Name | Sequence |
| 1 | SC DNA 1 | 5′-TCTTGCGTAT CTTCTCCACC TCAGCGCAAG ATCAACATCA GTCTGATAAG CTA-idT-idT-3′ |
| 2 | SC DNA 2 | 5′-TCTTGCGTAT CTTCTCCACC TCAGCGCAAG ATCAACAααA GTCTGATAAG CTA-idT-idT-3′ |
| 3 | SC DNA 3 | 5′-TCTTGCGTAT CTTCTCCACC TCAGCGCAAG ATCAACAββA GTCTGATAAG CTA-idT-idT-3′ |
| 4 | SC DNA 4 | 5′-TCTTGCGTAT CTTCTCCACC TCAGCGCAAG ATCAACAαCA GTCTGATAAG CTA-idT-idT-3′ |
| 5 | SC DNA 5 | 5′-TCTTGCGTAT CTTCTCCACC TCAGCGCAAG ATCAACAααα GTCTGATAAG CTA-idT-idT-3′ |
| 6 | SC DNA 6 | 5′-TCTTGCGTAT CTTCTCCACC TCAGCGCAAG ATCAACAααα αTCTGATAAG CTA-idT-idT-3′ |
| 7 | SC DNA 7 | 5′-TCTTGCGTAT CTTCTCCACC TCAGCGCAAG ATCAACATCα αTCTGATAAG CTA-idT-idT-3′ |
| 8 | SC DNA 8 | 5′-TCTTGCGTAT CTTCTCCACC TCAGCGCAAG ATCAACATαα GTCTGATAAG CTA-idT-idT-3′ |
| 9 | SC DNA 9 | 5′-TCTTGCGTAT CTTCTCCACC TCAGCGCAAG ATCAACααCA GTCTGATAAG CTA-idT-idT-3′ |
| 10 | SC DNA 10 | 5′-TCTTGCGTAT CTTCTCCACC TCAGCGCAAG ATCAAααTCA GTCTGATAAG CTA-idT-idT-3′ |
| 11 | SC DNA 11 | 5′-TCTTGCGTAT CTTCTCCACC TCAGCGCAAG ATCAααATCA GTCTGATAAG CTA-idT-idT-3′ |
| 12 | SA DNA 1 | 5′-AGCAGCAACA TAGCAGAACC TCAGCGCTGC TTCTTGCααA TCTTCTCCA-idT-idT-3′ |
| 13 | SC DNA 12 | 5′-AGCAGCAACA TAGCAGAACC TCAGCTTCTG CTATGTTGCT GCTTCAACAα αAGTCTGATA AGCTA-idT-idT-3′ |
| 14 | SA DNA 2 | 5′-TCTTGCGTAT CTTCTCCACC TCAGCATACG CAAGAAGCAG ααACATAGCA GAA-idT-idT-3′ |
| 15 | SC DNA 13 | 5′-AGCAGCAACA TAGCAGAACC TCAGCTTCTG CTATGTTGCT GCTTCAACAT AAGGTCTGATA AGCT-idT-idT-3′ |
| 16 | SA DNA 3 | 5′-TCTTGCGTAT CTTCTCCACC TCAGCATACG CAAGAAGCAG CAACATAGCA GAA-idT-idT-3′ |
| 17 | SC DNA 14 | 5′-TCTTGCGTAT CTTCTCCACC TCAGCATACG CAAGATCAACAα αAGTCTGATA AGCTA-idT-idT-3′ |
| 18 | SC DNA 15 | 5′-TCTTGCGTAT CTTCTCCACC TCAGCGAAGA TACGCAAGAT CAACAα αAGTCTGATA AGCTA-idT-idT-3′ |
| 19 | SC DNA 16 | 5′-TCTTGCGTAT CTTCTCCACC TCAGCTGGAG AAGATACGCA AGATCAACAα αAGTCTGATA AGCTA-idT-idT-3′ |
| 20 | model DNA | 5′-TAGCTTATCAGACTGATGTTGA-3′ |
| 21 | model RNA | 5′-UAGCUUAUCAGACUGAUGUUGA-3′ |

TABLE 2

| | | | | Sequence | | |
|---|---|---|---|---|---|---|
| | | | Enzyme | complementary | Sequence | Sequence |
| SEQ | | Signal DNA | recog- | to the signal | complementary to the | complementary to the |
| ID | | generation | nition | DNA generation | 3′-end of the target | 3′-end of the |
| NO. | Name | sequence | sequence | seq | nucleic acid sequence | Signal DNA sequence |
| 1 | SC DNA 1 | 5′-TCTTGCGTAT | CC TCAGC | GCAAG A | TCAACATCA GTCTGATAAG | |
| | | CTTCTCCA | | | CTA | |
| 2 | SC DNA 2 | 5′-TCTTGCGTAT | CC TCAGC | GCAAG A | TCAACAααA GTCTGATAAG | |
| | | CTTCTCCA | | | CTA | |
| 3 | SC DNA 3 | 5′-TCTTGCGTAT | CC TCAGC | GCAAG A | TCAACAββA GTCTGATAAG | |
| | | CTTCTCCA | | | CTA | |
| 4 | SC DNA 4 | 5′-TCTTGCGTAT | CC TCAGC | GCAAG A | TCAACAαCA GTCTGATAAG | |
| | | CTTCTCCA | | | CTA | |
| 5 | SC DNA 5 | 5′-TCTTGCGTAT | CC TCAGC | GCAAG A | TCAACAααα GTCTGATAAG | |
| | | CTTCTCCA | | | CTA | |
| 6 | SC DNA 6 | 5′-TCTTGCGTAT | CC TCAGC | GCAAG A | TCAACAααα αTCTGATAAG | |
| | | CTTCTCCA | | | CTA | |
| 7 | SC DNA 7 | 5′-TCTTGCGTAT | CC TCAGC | GCAAG A | TCAACATCα αTCTGATAAG | |
| | | CTTCTCCA | | | CTA | |
| 8 | SC DNA 8 | 5′-TCTTGCGTAT | CC TCAGC | GCAAG A | TCAACATαα GTCTGATAAG | |
| | | CTTCTCCA | | | CTA | |
| 9 | SC DNA 9 | 5′-TCTTGCGTAT | CC TCAGC | GCAAG A | TCAACααCA GTCTGATAAG | |
| | | CTTCTCCA | | | CTA | |
| 10 | SC DNA 10 | 5′-TCTTGCGTAT | CC TCAGC | GCAAG A | TCAAααTCA GTCTGATAAG | |
| | | CTTCTCCA | | | CTA | |
| 11 | SC DNA 11 | 5′-TCTTGCGTAT | CC TCAGC | GCAAG A | TCAααATCA GTCTGATAAG | |
| | | CTTCTCCA | | | CTA | |
| 12 | SA DNA 1 | 5′-AGCAGCAACA | CC TCAGC | GCTGCT | | TCTTGCααA TCTTCTCCA |
| | | TAGCAGAA | | | | |
| 13 | SC DNA 12 | 5′-AGCAGCAACA | CC TCAGC | TTCTG | TCAACAα αAGTCTGATA | |
| | | TAGCAGAA | | CTATGTTGCT GCT | AGCTA | |
| 14 | SA DNA 2 | 5′-TCTTGCGTAT | CC TCAGC | ATACG CAAGA | | AGCAG ααACATAGCA GAA |
| | | CTTCTCCA | | | | |
| 15 | SC DNA 13 | 5′-AGCAGCAACA | CC TCAGC | TTCTG | TCAACAT AAGTCTGATA | |
| | | TAGCAGAA | | CTATGTTGCT GCT | AGCTA | |
| 16 | SA DNA 3 | 5′-TCTTGCGTAT | CC TCAGC | ATACG CAAGA | | AGCAG CAACATAGCA GAA |
| | | CTTCTCCA | | | | |
| 17 | SC DNA 14 | 5′-TCTTGCGTAT | CC TCAGC | ATACG CAAGA | TCAACAα αAGTCTGATA | |
| | | CTTCTCCA | | | AGCTA | |
| 18 | SC DNA 15 | 5′-TCTTGCGTAT | CC TCAGC | GAAGA | TCAACAα αAGTCTGATA | |
| | | CTTCTCCA | | TACGCAAGA | AGCTA | |
| 19 | SC DNA 16 | 5′-TCTTGCGTAT | CC TCAGC | TGGAG | TCAACAα αAGTCTGATA | |
| | | CTTCTCCA | | AAGATACGCA AGA | AGCTA | |

While the application has been described with reference to certain aspects and embodiments, it will be understood by those skilled in the art that changes may be made to the disclosure provided herein, and equivalents may be substituted without departing from the scope of the disclosure. Accordingly, the application should not be limited to the particular aspects and embodiments disclosed, but should be understood and appreciated to include all aspect and embodiments falling within the scope of the appended claims.

1. A method for detecting a target nucleic acid in a sample, the method comprising contacting the sample with:
a first oligonucleotide comprising, in the 5′ to 3′ direction, a signal DNA generation sequence, an endonuclease recognition site, and a complementary sequence that comprises at least one abasic moiety and wherein the complementary sequence comprises a first complementary sequence that is complementary to at least a portion of the signal DNA generation sequence and a second complementary sequence that is complementary to the 3′ end of the target nucleic acid;
a polymerase; and
an endonuclease for a nicking reaction,
wherein the at least one abasic moiety is located between the 5′ end and the 3′ end of the second complementary sequence and is selected from a nucleotide position that is 3, 4, 5, 6, 7, 8, 9, 10, 11, 12, 13, 14, 15, nucleotides from the 5′ end of the second complementary sequence; and
wherein at least a portion of the signal DNA generation sequence and the first complementary sequence that is complementary to the portion of the signal DNA generation sequence hybridize to form a hairpin structure.

2. The method of claim 1, wherein the first oligonucleotide comprises a plurality of abasic moieties.

3. The method of claim 1, wherein the first oligonucleotide comprises an abasic moiety located at a position that is 7 nucleotides from the 5′ end of the second complementary sequence that is complementary to the 3′ end of the target nucleic acid.

4. The method of claim 2, wherein the first oligonucleotide comprises two abasic moieties located at positions that are 7 and 8 nucleotides from the 5′ end of the second complementary sequence that is complementary to the 3′ end of the target nucleic acid.

5. The method of claim 2, wherein the first oligonucleotide comprises three abasic moieties located at positions that are 7, 8 and 9 nucleotides from the 5′ end of the second complementary sequence that is complementary to the 3′ end of the target nucleic acid.

6. The method of claim 2, wherein the first oligonucleotide comprises four abasic moieties located at positions that are 7, 8, 9 and 10 nucleotides from the 5′ end of the second complementary sequence that is complementary to the 3′ end of the target nucleic acid.

7. The method of claim 1, wherein the method is performed at a substantially constant temperature.

8. The method of claim 1, wherein the method is performed at a temperature from about 20° C. to about 42° C.

9. The method of claim 1, wherein the polymerase has strand displacement activity.

10. The method of claim 1, wherein the polymerase is 3′ to 5′ exonuclease deficient, 5′ to 3′ exonuclease deficient, or both.

11. The method of claim 1, wherein the target nucleic acid is a micro-RNA.

12. The method of claim 1, wherein the target nucleic acid originates from an infectious agent.

13. The method of any of claims 1-12, wherein the method provides for 99% to 10% decrease in background signal amplification, relative to a control reaction that does not comprise an oligonucleotide incorporating one or more abasic modifications into the oligonucleotide sequence.

14. The method of claim 13, wherein the method reduces non-specific background signal amplification by about 30%.

15. The method of any of claims 1-14, wherein the first oligonucleotide comprises a 3′ end modification.

16. A composition for detecting a target nucleic acid in a sample, the composition comprising:
a first oligonucleotide comprising, in the 5′ to 3′ direction, a signal DNA generation sequence, an endonuclease recognition site, and a complementary sequence that comprises at least one abasic moiety and wherein the complementary sequence comprises a first complementary sequence that is complementary to at least a portion of the signal DNA generation sequence and a second complementary sequence that is complementary to the 3′ end of the target nucleic acid, and wherein at least a portion of the signal DNA generation sequence and the first complementary sequence that is complementary to the portion of the signal DNA generation sequence hybridize to form a hairpin structure.

17. The composition of claim 16, wherein the at least one abasic moiety is located between the 5′ end and the 3′ end of the second complementary sequence and is selected from a nucleotide position that is 3, 4, 5, 6, 7, 8, 9, 10, 11, 12, 13, 14, 15, nucleotides from the 5′ end of the second complementary sequence.

18. The composition of claim 16, wherein the first oligonucleotide comprises a plurality of abasic moieties.

19. The composition of claim 16, wherein the first oligonucleotide comprises an abasic moiety located at a position that is 7 nucleotides from the 5′ end of the second complementary sequence that is complementary to the 3′ end of the target nucleic acid.

20. The composition of claim 16, wherein the first oligonucleotide comprises two abasic moieties located at positions that are 7 and 8 nucleotides from the 5′ end of the second complementary sequence that is complementary to the 3′ end of the target nucleic acid.

21. The composition of claim 16, wherein the first oligonucleotide comprises three abasic moieties located at positions that are 7, 8 and 9 nucleotides from the 5′ end of the second complementary sequence that is complementary to the 3′ end of the target nucleic acid.

22. The composition of claim 16, wherein the first oligonucleotide comprises four abasic moieties located at positions that are 7, 8, 9 and 10 nucleotides from the 5′ end of the second complementary sequence that is complementary to the 3′ end of the target nucleic acid.

23. The composition of claim 16, further comprising a polymerase and an endonuclease for a nicking reaction.

24. The composition of claim 23, wherein the polymerase has strand displacement activity.

25. The composition of claim 23, wherein the polymerase is 3′ to 5′ exonuclease deficient, 5′ to 3′ exonuclease deficient, or both.

26. The composition according to any of claims 16-25, wherein the first oligonucleotide comprises a 3′ end modification.

27. A kit for detecting a target nucleic acid in a sample, the kit comprising:
a first oligonucleotide comprising, in the 5′ to 3′ direction, a signal DNA generation sequence, an endonuclease recognition site, and a complementary sequence that comprises at least one abasic moiety and wherein the complementary sequence comprises a first complementary sequence that is complementary to at least a portion of the signal DNA generation sequence and a second complementary sequence that is complementary to the 3′ end of the target nucleic acid, wherein the at least one abasic moiety is located between the 5′ end and the 3′ end of the second complementary sequence and is selected from a nucleotide position that is 3, 4, 5, 6, 7, 8, 9, 10, 11, 12, 13, 14, 15, nucleotides from the 5′ end of the second complementary sequence, and wherein at least a portion of the signal DNA generation sequence and the first complementary sequence that is complementary to the portion of the signal DNA generation sequence hybridize to form a hairpin structure.

28. A chemically modified oligonucleotide comprising, in the 5′ to 3′ direction, a signal DNA generation sequence, an endonuclease recognition site, and a complementary sequence that comprises at least one abasic moiety; wherein the complementary sequence comprises a first complementary sequence that is complementary to at least a portion of the signal DNA generation sequence and a second complementary sequence that is complementary to the 3′ end of a target nucleic acid; wherein the at least one abasic moiety is located between the 5′ end and the 3′ end of the second complementary sequence and is selected from a nucleotide position that is 3, 4, 5, 6, 7, 8, 9, 10, 11, 12, 13, 14, 15, nucleotides from the 5′ end of the second complementary sequence; and wherein at least a portion of the signal DNA generation sequence and the first complementary sequence that is complementary to the portion of the signal DNA generation sequence hybridize to form a hairpin structure.

29. The chemically modified oligonucleotide of claim 28, wherein the oligonucleotide comprises a plurality of abasic sites.

30. The chemically modified oligonucleotide of claim 28, wherein the oligonucleotide comprises an abasic moiety located at a position that is 7 nucleotides from the 5′ end of the second complementary sequence that is complementary to the 3′ end of the target nucleic acid.

31. The chemically modified oligonucleotide of claim 29, wherein the oligonucleotide comprises two abasic moieties located at positions that are 7 and 8 nucleotides from the 5′ end of the second complementary sequence that is complementary to the 3′ end of the target nucleic acid.

32. The chemically modified oligonucleotide of claim 29, wherein the oligonucleotide comprises three abasic moieties located at positions that are 7, 8 and 9 nucleotides from the 5′ end of the second complementary sequence that is complementary to the 3′ end of the target nucleic acid.

33. The chemically modified oligonucleotide of claim 29, wherein the oligonucleotide comprises four abasic moieties located at positions that are 7, 8, 9 and 10 nucleotides from the 5′ end of the second complementary sequence that is complementary to the 3′ end of the target nucleic acid.

34. The chemically modified oligonucleotide according to any of claims 28-33, further comprising a 3′ end modification.
